(19) United States
(12) Patent Application Publication
(10) Pub. No.: US 2023/0190819 A1
(43) Pub. Date: Jun. 22, 2023

(54) COMPOSITIONS AND METHODS CONTAINING EXOSOMES

(71) Applicant: YOUNGSUK YI, POTOMAC, MD (US)

(72) Inventor: YOUNGSUK YI, POTOMAC, MD (US)

(21) Appl. No.: 17/055,573

(22) PCT Filed: May 6, 2020
(86) PCT No.: PCT/US20/31685

§ 371 Date: Nov. 14, 2020

Related U.S. Application Data
(60) Provisional application No. 62/846,622, filed on May 11, 2019.

(51) Int. Cl. A61K 35/32; A61K 47/42; A61K 38/18; A61K 35/33; A61K 35/30; C12N 5/0793; C12N 5/079

(52) U.S. Cl. CPC A61K 35/32; A61K 47/42; A61K 38/1841; A61K 35/33; A61K 35/30; C12N 5/0619; C12N 5/0622; C12N 2506/45; C12N 2506/1353

(57) ABSTRACT

Disclosed herein is a technology platform for delivering macromolecules of interest to a subject over a period of time without repeated administration. The compositions disclosed herein are comprised of genetically modified cells, engineered exosomes, and adhesive scaffold materials. The compositions are designed to sustain the supply macromolecules of interest at a site of administration for a long period of time ranging from days to months.

### CROSS-REFERENCE

This application claims the benefit of PCT Application PCT/US20/31685, filed on May 6, 2020, which claims the benefit of U.S. Provisional Application No. 62/846,622, filed May 11, 2019, which application is incorporated herein by reference in its entirety.

### BACKGROUND OF THE INVENTION

Exosomes are membrane-bound vesicles. They differ from other vesicles based on their biogenesis and biophysical properties, including size and surface protein markers. Exosomes are homogenous small particles ranging from 40 to 300 nm in size.

Some exosomes are derived from the endocytic recycling pathway. Generally, small, endocytic vesicles are formed via the fusion of the plasma membrane. As the vesicles mature, they become endosomes where intraluminal vesicles bud off into an intra-vesicular lumen, forming multi-vesicular bodies. They then directly fuse with the plasma membrane, and exosomes are released into the extracellular space.

The endosomal sorting complex required for transport (ESCRT complex) regulates the exosome biogenesis. Through the ESCRT-dependent or independent pathways, exosomes are formed and packaged within endosomes using sphingolipids and tetraspanins. Endosomes formed in this way are either digested by entering the lysosomal pathway, or fused with the plasma membrane and thereby releasing the exosomes within the endosomes. Exosomes contain a variety of biological material, including, but are not limited to, miRNAs, mRNAs, proteins such as Alix, Tsg101, I-integrin, MFG-E8, ICAM-1, the membrane markers such as CD63 and CD81, or a combination thereof. To date, about 134 studies on exosomes have reported 4,049 proteins, 1,639 mRNA molecules, 58 lipid molecules, and 764 miRNA molecules.

Exosomes function as signaling modalities, capable of modifying the phenotype of recipient cells. As a vehicle for delivering cargo molecules between cells, exosomes can directly mediate the transfer of biological material between cells. Exosomes can trigger a signal transduction pathway by providing a ligand to a molecule at cell surface, or change the molecular composition of the recipient cells. In addition, exosomes are involved in other cellular processes such as antigen presentation, cell death, angiogenesis, inflammation, and coagulation.

Exosomes can function as a communication modality among different organs, as they can travel via the blood or cerebrospinal fluid. In the CNS, exosomes are involved in the crosstalk between neurons and glia. It has been reported that release of exosomes may affect synaptic activities by providing RNAs, proteins or lipids that function as signaling molecules. It has also been reported that exosomes are involved in the maintenance of myelination.

Exosomes secreted by neurons can deliver neuron-derived cargo specifically to other neuronal cells, such as astrocytes. Neuronal exosomes carrying miR-124a was found to be associated with enhanced expression of glutamate transporter-1 (GLT1), suggesting the exosome may influence glutamate uptake in the astrocytes by enhancing the expression of GLT1. Glial exosomes have been shown to be neurotrophic and neuroprotective. Astrocytes secretes exosomes in response to stress, and these exosomes have been shown to transfer pro-survival factors such as HSP70, Synapsin-I and LIF.

The skin of mammals consists of epidermis and dermis. The epidermis is an upper, multi-layered, stratified epithelium. The dermis is a thick layer of connective tissue underneath the epidermis. The cells in the epidermis are continuously replenished with cells differentiated from stem cells and/or progenitor cells in the basal layer of the epidermis. Below the basal layer is the basement membrane, which is consisted of extracellular proteins, that connects the epidermis with the dermis.

The basal-layer cells are anchored to the basement membrane through highly specialized multiprotein structures called hemidesmosomes. One of the main components of hemidesmosomes is Collagen type 17, encoded by COL17A1 gene. Reduced levels of Col17A1 are a hallmark of aging skin.

As animals age, the number of hemidesmosomes diminishes. This reduction is correlated with low levels of Col17A1. Loss of Col17A1 destabilizes hemidesmosomes, preventing the formation of functional hemidesmosomes. Col17A1 rapidly degrades in response to several types of stress, including exposure to ultraviolet light.

The epidermal stem cells in the basal-layer express various levels of Col17A1. As animals age, more epidermal stem cells that express high levels of Col17A1 is found in the basal layer of aged animal than that of a younger animal. As these high-level Col17A1 cells become dominant, there are fewer different stem cells (and hence, less diversity) in aged animal.

When Col17A1 expression is artificially reduced under experimental condition, the cells are detached from the basement membrane, and replaced with cells expressing high levels of Col17A1.

Cells in the basal layer divide in two different ways: divisions on a plane parallel to that of the basement membrane occur for replacement of old cells while divisions on a plane perpendicular to that of the basement membrane occur to produce differentiated epidermal cells of the non-basal layers of the skin. High levels of Col17A1 in epidermal stem cells result in “parallel” cell divisions, resulting in maintaining the number of stem cells in the basal layer. Loss of Col17A1 results in “perpendicular” cell divisions. As perpendicular cell divisions continue without parallel cell divisions, the pool of stem cells is depleted, resulting in clinical manifestations typically associated with aging including skin thinning and depigmentation. It has been demonstrated that restoring Col17A1 expression in the basal layer mitigates skin aging.

1-(4-Hydroxy-3-methoxyphenyl)ethan-1-one, also known as apocynin or acetovanillone, is a natural organic compound isolated from a variety of plant. Apocynin is a solid with a melting point of 115° C. and has a faint odor of vanilla. It is soluble in hot water, alcohol, benzene, chloroform, and ether.

Apocynin was first isolated by Horace Finnemore in 1908 from the root of Canadian hemp Apocynum cannabinum. At the time, this plant was already used for its known effectiveness against edema and heart problems. In 1971, apocynin was also isolated from Picrorhiza kurroa, a small plant that grows at high altitudes in the western Himalayas. P. kurroa was used for ages as a treatment for liver and heart problems, jaundice, and asthma. Apocynin's observed anti-inflammatory capabilities proved to be a result of its ability to selectively prevent the formation of free radicals, oxygen ions, and peroxides in the body.

NADPH oxidase is an enzyme that effectively reduces O2 to superoxide (O2X), which can be used by the immune system to kill bacteria and fungi. Apocynin is an inhibitor of NADPH oxidase activity and thus is effective in preventing the production of the superoxide in human white blood cells or neutrophilic granulocytes. It does not obstruct the phagocytic or other defense roles of granulocytes. Due to the selectivity of its inhibition, apocynin can be widely used as an inhibitor of NADPH oxidase without interfering in other aspects of the immune system.

Apocynin was used to determine whether ionic activation due to proton flux across the membrane of renal medulla cells was coupled to NADPH oxidase production of superoxide. Apocynin was introduced to the cells and completely blocked the production of superoxide, and was a key component in determining that the proton outflow was responsible for the activation of NADPH oxidase.

Apocynin is shown to dimerize and form diapocynin. Biotransformation of apocynin predominantly leads to glycosylated form of apocynin.

(1R,4r)-4-((R)-1-aminoethyl)-N-(pyridin-4-yl)cyclohexanecarboxamide, known as Y-27632 is a is a selective inhibitor of p160ROCK.

Mesenchymal stem cells (MSCs) are multipotent stromal cells that can differentiate into a variety of cell types, including osteoblasts, chondrocytes, myocytes, and adipocytes. MSCs have a great capacity for self-renewal while maintaining their multipotency. The cultured MSCs also express on their surface CD73, CD90 and CD105, while lacking the expression of CD11b, CD14, CD19, CD34, CD45, CD79a and HLA-DR surface markers. MSCs do not differentiate into hematopoietic cells. The term MSCs encompasses multipotent cells derived from other non-marrow tissues, such as placenta umbilical cord blood, adipose tissue, adult muscle, corneal stroma, or the dental pulp of deciduous baby teeth. MSCs do not have the capacity to reconstitute an entire organ. The standard test to confirm multipotency is differentiation of the cells into osteoblasts, adipocytes and chondrocytes as well as myocytes and neurons.

Human bone marrow derived MSCs show fibroblast-like morphology. MSCs are characterized morphologically by a small cell body with a few cell processes that are long and thin. The cells, which are long and thin, are widely dispersed and the adjacent extracellular matrix is populated by a few reticular fibrils but is devoid of the other types of collagen fibrils.

Bone marrow is a source of MSCs. These bone marrow MSCs do not contribute to the formation of blood cells and so do not express the hematopoietic stem cell marker CD34. The youngest and most primitive MSCs may be obtained from umbilical cord tissue, namely Wharton's jelly and the umbilical cord blood. However MSCs are found in much higher concentration in the Wharton's jelly compared to cord blood, which is a rich source of hematopoietic stem cells. Adipose tissue is a rich source of MSCs. The developing tooth bud of the mandibular third molar is a rich source of MSCs. Dental-pulp MSCs are capable of producing hepatocytes. MSCs are present in amniotic fluid. As many as 1 in 100 cells collected during amniocentesis are pluripotent MSCs.

MSCs have an effect on innate and specific immune cells. MSCs produce many molecules having immunomodulatory effects. These include prostaglandin E2 (PGE2), nitric oxide, indolamin 2,3-dioxigenase (IDO), IL-6, and other surface markers—FasL, PD-L1/2. MSCs have an effect on macrophages, neutrophils, NK cells, mast cells and dendritic cells in innate immunity. Macrophage phenotype is divided into two groups: the M1 phenotype has a pro-inflammatory effect, and the M2 has anti-inflammatory effect. MSCs are able to migrate to the site of injury where they produce PGE2, which in turn polarize macrophages to the M2 phenotype, initiating the anti-inflammatory effect. Further, PGE2 inhibits the ability of mast cells to degranulate and produce TNF-alpha. Proliferation and cytotoxic activity of NK cells is inhibited by PGE2 and IDO. MSCs also reduce the expression of NK cell receptors—NKG2D, NKp44 and NKp30. MSCs inhibit respiratory flare and apoptosis of neutrophils by production of cytokines IL-6 and IL-8. Differentiation and expression of dendritic cell surface markers is inhibited by IL-6 and PGE2 of MSCs. The immunosuppressive effects of MSC also depend on IL-10. MSC expresses the adhesion molecules VCAM-1 and ICAM-1, which allow T-lymphocytes to adhere to their surface. Then MSC can affect them by molecules which have a short half-life and their effect is in the immediate vicinity of the cell. These include nitric oxide, PGE2, HGF, and activation of receptor PD-1. MSCs reduce T cell proliferation between G0 and G1 cell cycle phases and decrease the expression of IFN-gamma of Th1 cells while increasing the expression of IL-4 of Th2 cells. MSCs also inhibit the proliferation of B-lymphocytes between G0 and G1 cell cycle phases. MSCs can produce antimicrobial peptides (AMPs). These include human cathelicidin LL-37, beta-defensines, lipocalin 2, and hepcidin. MSCs effectively decrease number of colonies of both gram negative and gram positive bacteria by production of these AMPs. In addition, the same antimicrobial effect of the enzyme IDO produced by MSCs was found. MSCs in the body can be activated and mobilized if needed. However, the efficiency is low.

### SUMMARY OF THE INVENTION

In one aspect, described herein is a composition comprising: differentiated human cells; adhesive scaffold material; and engineered exosomes carrying cargo molecules.

In one embodiment, said cargo molecules are Col17A1 activator, apocynin, Y-27632, Col17A1, TGF-β1, TGF-β2, TGF-β3, BMP-2, BMP-3, BMP-4, BMP-5, BMP-6, BMP-7, BMP-9 or a combination thereof.

In one embodiment, about 50 percent of said cargo molecules are encapsulated within said exosomes, and about 50 percent of the rest of said cargo molecules are not bound to, or encapsulated within said exosomes.

In one embodiment, substantially all of said cargo molecules are encapsulated within said exosomes.

In one embodiment, said exosomes are isolated from keratinocytes, thymic epithelial cells, induced pluripotent stem cell, or mesenchymal stem cells.

In one embodiment, said exosomes are derived from 1OT1/2, BALB/3T3, L-M, NB4, 1A3, NIE-1 15, NG108-15, NIH3T3, NCTC, Neuro-2A, PC12, GH1, GH3, C6, L2, CHO, OHO, 6E6, PK15, LLC-PK1, ST, ESK-4, CPAE, BT, FB2, SBAC, NBL-6, COS-1, COS-7, or VV-1, SH-SY5Y, SK-N-DZ, SK-N-F1, SK-N-SH, BE(2)-C, HeLa, HEK 293, MCF-7, HepG2, HL-60, IMR-32, SW-13, OHP3, or CHPS.

In one embodiment, said exosomes encapsulate said cargo by passive diffusion, sonication, saponication, heating, emulsification, freezing and thawing, or use of solvents.

In one embodiment, said exosomes are isolated from cells genetically modified to express said cargo molecules.

In one embodiment, said exosomes are isolated from exosome secreting cells by ultracentrifugation, ultrafiltration, size-exclusion chromatography, immunoisolations, precipitation, or microfluidics-based isolation.

In one embodiment, said exosomes have Synapsin I.

In one embodiment, said cargo molecules fills up about 30% of the lumen of said exosomes.

In one embodiment, said differentiated cells are derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, or precursor cells.

In one embodiment, the amount of said differentiated cells are 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

In one embodiment, said adhesive scaffold material is porous silk fibroin wherein said scaffold has pores of 50 to 1,000 microns in diameter, a density of 20-200 m/ml, a porosity from 50-99.5%, and a compressive modulus of at least 100 kPa.

In one embodiment, said adhesive scaffold material is cell-based extracellular matrix or buffy coat.

In one embodiment, said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

In one embodiment, said composition further comprise a buffered solution selected from the group consisting of phosphate-buffered saline, Hanks balance salt solution, or saline.

In one embodiment, said cargo molecules are released in biphasic manner in which about 50 percent of the cargo molecules are released during the first phase and the other 50 percent of the cargo molecules are released during the second phase.

In one embodiment, the amount of said exosomes in a package is about 4 mg.

In one aspect, described herein is a composition comprising 1×10⁶ human chondrocytes; exosomes carrying TGF-β1, and an adhesive scaffold material.

In one embodiment, said composition further comprising TGF-β1.

In one embodiment, said scaffold material is buffy coat.

In one aspect, described herein is a composition comprising 1×10⁶ human fibroblasts; exosomes carrying apocynin, and an adhesive scaffold material.

In one embodiment, said scaffold material is collagen matrix.

In one aspect, described herein is a method comprising differentiating mammalian cells in vitro; culturing exosome secreting cells; modifying said exosome-producing cells or exosomes secreted therefrom to carry cargo molecules in said exosome; isolating said exosomes; and formulating an injectable composition comprising said differentiated cells, said exosomes, and adhesive scaffold material.

In one embodiment, said cargo molecules are Col17A1 activator, apocynin, Y-27632, Col17A1, TGF-β1, TGF-β2, TGF-β3, BMP-2, BMP-3, BMP-4, BMP-5, BMP-6, BMP-7, BMP-9, or a combination thereof.

In one embodiment, said injectable composition has about 50 percent of said cargo molecules encapsulated within said exosomes, and about 50 percent of the rest of said cargo molecules not bound to, or encapsulated within said exosomes.

In one embodiment, substantially all of said cargo molecules are encapsulated within said exosomes.

In one embodiment, said mammalian cells are keratinocytes, thymic epithelial cells, induced pluripotent stem cell, or mesenchymal stem cells.

In one embodiment, said modifying is passive diffusion, sonication, saponication, heating, emulsification, freezing and thawing, or use of solvents.

In one embodiment, said modifying is genetically modifying said exosome secreting cells to express said cargo molecules.

In one embodiment, said isolating is ultracentrifugation, ultrafiltration, size-exclusion chromatography, immunoisolations, precipitation, or microfluidics-based isolation.

In one embodiment, said exosomes have Synapsin I.

In one embodiment, said cargo molecules fills up about 30% of the lumen of said exosomes.

In one embodiment, said differentiated cells are derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, induced pluripotent stem cells, or precursor cells.

In one embodiment, the amount of said differentiated cells are 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

In one embodiment, said adhesive scaffold material is porous silk fibroin wherein said scaffold has pores of 50 to 1,000 microns in diameter, a density of 20-200 m/ml, a porosity from 50-99.5%, and a compressive modulus of at least 100 kPa.

In one embodiment, said adhesive scaffold material is cell-based extracellular matrix or buffy coat.

In one embodiment, said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

In one embodiment, said injectable composition further comprise a buffered solution selected from the group consisting of phosphate-buffered saline, Hanks balance salt solution, or saline.

In one embodiment, said cargo molecules are released in biphasic manner in which about 50 percent of the cargo molecules are released during the first phase and the other 50 percent of the cargo molecules are released during the second phase.

In one embodiment, the amount of said exosomes in said injectable composition is about 4 mg.

In one aspect, described herein is a method of identifying compositions carrying therapeutic amounts of engineered exosomes, differentiated cells, and adhesive scaffold material comprising: culturing exosome-secreting cells; modifying said cells or exosomes secreted therefrom to carry cargo molecules in said exosome; isolating said exosomes; formulating an injectable composition comprising said exosomes, differentiated cells, adhesive scaffold material, and pharmaceutically acceptable excipients; administering said formulation to a human subject; examining the clinical responses of a human subject; and identifying formulations with desired therapeutic effects.

In one embodiment, said cargo molecules are Col17A1 activator, apocynin, Y-27632, Col17A1, TGF-β1, TGF-β2, TGF-β3, BMP-2, BMP-3, BMP-4, BMP-5, BMP-6, BMP-7, BMP-9, or a combination thereof.

In one embodiment, said injectable composition has about 50 percent of said cargo molecules encapsulated within said exosomes, and about 50 percent of the rest of said cargo molecules not bound to, or encapsulated within said exosomes.

In one embodiment, substantially all of said cargo molecules are encapsulated within said exosomes.

In one embodiment, said culturing is growing keratinocytes, thymic epithelial cells, induced pluripotent stem cell, or mesenchymal stem cells in vitro.

In one embodiment, said modifying is passive diffusion, sonication, saponication, heating, emulsification, freezing and thawing, or use of solvents.

In one embodiment, said modifying is genetically modifying said exosome secreting cells to express said cargo molecules.

In one embodiment, said isolating is ultracentrifugation, ultrafiltration, size-exclusion chromatography, immunoisolations, precipitation, or microfluidics-based isolation.

In one embodiment, said exosomes have Synapsin I.

In one embodiment, said cargo molecules fills up about 30% of the lumen of said exosomes.

In one embodiment, said differentiated cells are derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, or precursor cells.

In one embodiment, the amount of said differentiated cells are 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

In one embodiment, said adhesive scaffold material is porous silk fibroin wherein said scaffold has pores of 50 to 1,000 microns in diameter, a density of 20-200 m/ml, a porosity from 50-99.5%, and a compressive modulus of at least 100 kPa.

In one embodiment, said adhesive scaffold material is cell-based extracellular matrix or buffy coat.

In one embodiment, said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

In one embodiment, said injectable composition further comprise a buffered solution selected from the group consisting of phosphate-buffered saline, Hanks balance salt solution, or saline.

In one embodiment, said cargo molecules are released in biphasic manner in which about 50 percent of the cargo molecules are released during the first phase and the other 50 percent of the cargo molecules are released during the second phase.

In one embodiment, the amount of said exosomes in said injectable composition is about 4 mg.

In one embodiment, said clinical response is measuring VAS, KOOS, or IKDC scoring.

In one embodiment, said desired effect is a change in clinical response recorded by at least one unit of measure employed in a scoring method.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates delivery of one embodiments of the present invention to a site of injury. A composition of the present invention 3 is loaded into a syringe 1 and injected through the skin 2. The dropwise application of the present invention forms an adhesive scaffold 4 and the content of the present invention 3 is released to the site of injury 5.

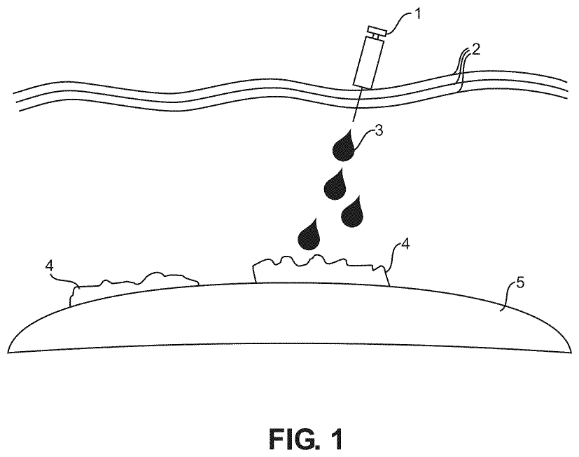

FIG. 2 illustrates a composition of the present invention 3 in a formulation comprising differentiated cells 4, adhesive scaffold material 6, exosomes 7 containing cargo molecules 5.

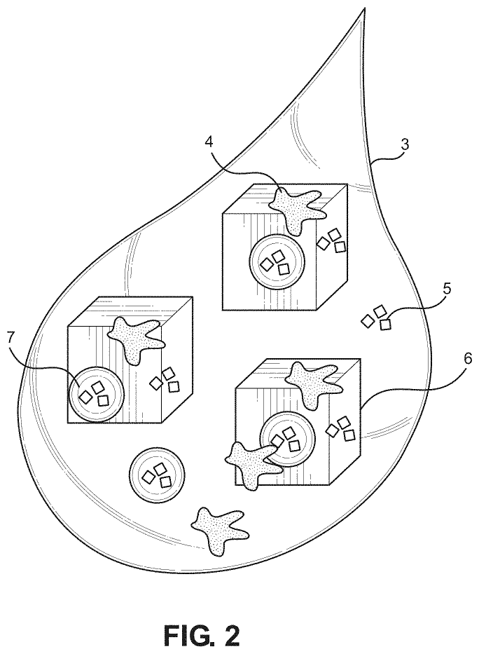

FIG. 3 illustrates a method of selecting exosomes suitable for therapeutic use in human

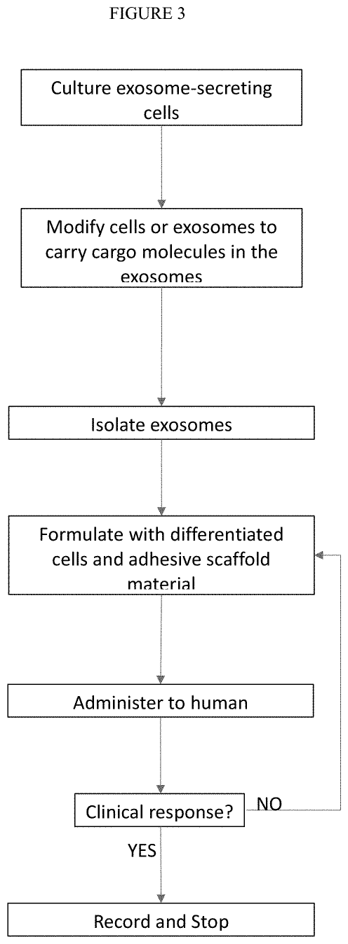

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions/Nomenclature

As used herein unless otherwise indicated, open terms such as “contain,” “containing,” “include,” “including,” and the like mean comprising.

Some embodiments herein contemplate numerical ranges. When a numerical range is provided, the range includes the range endpoints unless otherwise indicated. Unless otherwise indicated, numerical ranges include all values and subranges therein as if explicitly written out.

Some values herein are modified by the term “about.” In some instances, the term “about” in relation to a reference numerical value can include a range of values plus or minus 10% from that value. For example, the amount “about 10” can include amounts from 9 to 11. In other embodiments, the term “about” in relation to a reference numerical value can include a range of values plus or minus 10%, 9%, 8%, 7%, 6%, 5%, 4%, 3%, 2%, or 1% from that value.

As used herein, the article “a” means one or more unless explicitly stated otherwise.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “×g” is multiples of g, the standard acceleration due to gravity at the Earth's surface, “min” means minute(s), “kPa” means kilopascal, “C” means Celsius or degrees Celsius, as is clear from its usage, “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “uT” means microtesla, “g” means gram(s), “μg” or “ug” means microgram(s) and “ng” means nanogram(s), “% w/v” means weight/volume percent, “% v/v” means volume/volume percent, “HPLC” means high-performance liquid chromatography, “UPLC” means ultra-performance liquid chromatography, and “GC” means gas chromatography.

Compositions and methods described herein provide for engineered exosomes useful for carrying macromolecules, chemical compound, or both, as a cargo; differentiated human cells; and adhesive scaffold material. In accordance with one aspect of the invention, the engineered exosomes release the cargo by fusing with recipient cells. In another aspect, the engineered exosomes described herein pass through the human skin via permeation. In another aspect, the compositions described herein are administered to a site of injury.

The engineered exosomes of the present invention are manufactured by modifying the exosome-secreting cells or by modifying naturally-occurring exosomes. Both methods require selecting one or more exosome secreting cells as the first step of the manufacturing process. Cells originated from any one of the three germ layers have been found to produce exosomes. Thus, virtually any type of cells are useful for the present invention as a source of exosome secreting cells.

Primary cells useful for the present invention can be derived from the endoderm, the mesoderm, or the ectoderm. Cells derived from the endoderm include, but are not limited to, cells of the respiratory system, the intestine, the liver, the gallbladder, the pancreas, the islets of Langerhans, the thyroid, or the hindgut. Cells derived from the mesoderm include, but are not limited to, cells of the osteochondroprogenitor cells, the muscle, the digestive systems, the renal system such as renal stem cells, the reproductive system, bloods cells, or cells from the circulatory system, such as endothelial cells. Cells derived from the ectoderm include, but are not limited to, epithelial cells, cells of the anterior pituitary, cells of the peripheral nervous system, cells of the neuroendocrine system, cell of the teeth, cell of the eyes, cells of the central nervous system, cells of the ependymal, or cells of the pineal gland.

Primary cells of the central and the peripheral nervous system can be a source of exosome-secreting cells. These cells include, but are not limited to, Schwann cells, satellite glial cells, oligodendrocytes, or astrocytes. The neurons as used herein include, but are not limited to, peripheral neurons, interneurons, pyramidal neurons, gabaergic neurons, dopaminergic neurons, serotoninergic neurons, glutamatergic neurons, motor neurons from the spinal cord, or inhibitory spinal neurons.

Stem/progenitor cells are a special type of primary cells capable of renewing itself and/or differentiating into various cell types. Stem/progenitor cells of various origins have been found to produce exosomes. Stem/progenitor cells useful for the present invention can be isolated from umbilical-cord, umbilical cord blood, peripheral blood, embryo, adipose tissue, bone-marrow, hair-follicle, hair follicle, or the subgranular zone (SGZ) of the dentate gyms (DG) in the hippocampus and the subventricular zone (SVZ) in the forebrain. In one embodiment, stem cells useful for the present invention are mesenchymal stem cells (MSCs).

As to the neural stem/progenitor cells, the developing, as well as the adult central nervous system (CNS), contain multipotent cells with unlimited self-renewal referred to as neural stem/progenitor cells (NSCs). NSCs make a vital contribution to the formation of the CNS during development as they generate neurons, astrocytes, and oligodendrocytes, the three major cell types in the CNS. The primary NSCs are a subtype of radial glial cells that generate transit amplifying cells or intermediate progenitors through asymmetric cell division. The intermediate progenitors comprise the more restricted neuronal and glial progenitor cells displaying limited self-renewal and proliferative activity to produce differentiated neurons and glia. In the postnatal and adult periods, only a few areas in the CNS display active NSCs proficient in generating neurons, astrocytes, and oligodendrocytes. These regions, known as neurogenic regions, include the subgranular zone (SGZ) of the dentate gyms (DG) in the hippocampus and the subventricular zone (SVZ) in the forebrain.

Another source of exosome secreting cells useful for the present invention is induced pluripotent stem cells (iPSCs). Forced expression of Oct3/4, Sox2 and Klf4 (as well as OCT3/4, SOX2 and KLF4) is sufficient to generate an induced pluripotent stem cell out of an adult somatic cell, such as a fibroblast. Also, the combination of Oct3/4, Sox2, c-Myc and Klf4 (as well as OCT3/4, SOX2, C-MYC) and KLF4 can be sufficient for the generation of a iPSC from an adult somatic cell. In addition, the combination of OCT3/4, SOX2, NANOG and LIN28 can be efficient for reprogramming. These genes are cloned into a retroviral vector and transgene-expressing viral particles or vectors, with which the somatic cell is co-transduced. Human skin fibroblasts can also be co-transduced with all four vectors e.g. via protein transduction or naked DNA. iPSCs may be obtained from any adult somatic cell. Exemplary somatic cells include peripheral blood mononuclear cells (PBMCs) from blood or fibroblasts, such as fibroblasts obtained from skin tissue biopsies.

iPSCs described herein can be derived from adult human cells. Examples of human cells include, but are not limited to, adult dermal fibroblasts, fetal FRCS lung fibroblasts, or newborn BJ-1 foreskin fibroblasts. Systems useful for expressing the genes driving the reprogramming of adult cells to iPSCs include, but are not limited to, viral traduction methods using adenovirus, retrovirus, lentivirus, or sendai virus, or non-viral transfection methods using mRNA transfection, miRNA transfection, transfection of transposons, minicircle vectors, or episomal plasmids. Some material known to be useful for increasing the reprogramming efficiencies include, but are not limited to, hTERT, SV 40 large T antigen, a histone deacetylase inhibitor such as valproic acid or sodium butyrate, a MEK inhibitor such as PD0325901, a TGF-beta inhibitor such as A-82-01, SB43152, an epigenetic modifier such as vitamin C, a ROCK inhibitor such as thiazovivin, a PI3K inhibitor such as PS48, or a glycolysis promotor such as 5% oxygen. iPSCs described herein can be derived from human cells using one or more of the factors known to be useful for increasing the reprogramming efficiencies.

Cell lines are also known source of exosomes. Cell lines useful for the present invention can be derived from the primary cells and stem/progenitor cells described herein, or can be purchased from commercial providers, such as American Type Culture Collection (ATCC). Non-limiting examples of the established cell lines include Mus musculus cell line 1OT1/2, BALB/3T3, L-M, NB4, 1A3, NIE-1 15, NG108-15, NIH3T3, NCTC, or Neuro-2A, Rattus norvegicus cell line PC12, GH1, GH3, C6, or L2, Cricetulus griseus cell line CHO, OHO, or 6E6, Sus scrofa cell line PK15, LLC-PK1, ST, or ESK-4, Bos Taurus cell line CPAE, BT, FB2, or SBAC, Equus caballus cell line NBL-6, Cercopithecus aethiops cell line COS-1, COS-7, or VV-1, or Homo sapiens cell line HFF (Human foreskin fibroblasts), HDFa (normal human primary dermal fibroblasts), SH-SY5Y, SK-N-DZ, SK-N-F1, SK-N-SH, BE(2)-C, HeLa, HEK 293, MCF-7, HepG2, HL-60, IMR-32, SW-13, OHP3, or CHPS.

In some embodiments, the exosome-secreting cells useful for the present invention are thymic epithelial cells (TECs). Exosomes derived from TECs of the present invention carry one or more macromolecules including, but are not limited to, Collagen type II, Desmoglein 1, Desmoglein 3, Alpha-enolase, Titin, Laminin gamma 1, Collagen 17, Vinculin, Galectin 1, Galectin 3, Galectin 3, Heat shock protin 60, Tissue transglutaminase 2, or Myelin basic protein.

Keratinocytes secrete exosomes useful for releasing cargo molecules in fibroblasts. In one embodiment, keratinocytes or a cell line derived from keratinocytes are used to secrete engineered exosomes of the present invention.

In embodiments utilizing primary cells as a source of exosome, the exosome-secreting cells are isolated from the tissue of origin, and cultured in vitro to secrete exosomes. Methods of isolating and culturing primary cells are provided in the examples herein.

Once culture of exosome-secreting cells is established, the cells, exosomes, or both are modified to produce the engineered exosomes of the present invention.

In the present invention, the exosome-secreting cells are modified by genetic engineering methods including, but are not limited to, transfection, transduction, or a combination thereof. By these methods, the exosome-secreting cells are modified with plasmids to drive the expression of one or more cargo molecules. The cells can also be modified by electroporation, or by the use of a transfection reagent, to load the cargo molecules directly to the cells. Methods of modifying the exosome-secreting cells are provided in the examples herein.

The exosome-secreting cells of the present invention are modified to carry macromolecules. The macromolecules can be extracellular factors, manifesting its effect via an autocrine or paracrine route, or intracellular factors, manifesting its effect on the genome, or by influencing cellular signal transduction pathways . These macromolecules include, but are not limited to, Col17A1, TGF-β1, TGF-β2, TGF-β3, BMP-2, BMP-3, BMP-4, BMP-5, BMP-6, BMP-7, or a combination thereof. These macromolecules can be carried in the lumen or on the surface (as a transmembrane protein) of the exosomes derived from the exosome-secreting cells. TGF-β1, for example, is known to be present on the surface of an exosomes derived from mast cells. As another example, BMP-2 is present in exosomes secreted by mesenchymal stem cells derived from acute myeloid leukemia patient. Macromolecules on the surface of the exosomes can contact their natural receptors during the endocytosis, triggering the signal transduction pathways emanating from the receptors. In mesenchymal stem cells, TGF signaling pathway can be triggered during the endocytosis of exosomes via TGF-β1 on the surface of exosomes.

These macromolecules can also be directly loaded to the exosome-secreting cells by electroporating or transfecting the cells with purified proteins of these macromolecules. In one embodiment, the exosome-secreting cells of the present invention are electroporated or transfected with the one or more of the following macromolecules: alpha chain of type XVII collagen, fibrillar collagen such as Type I, II, III, V, XI collagen, non-fibrillar collagen such as type IV, VI, VII, IX, X, XII, XIII, XIV, XV, XIX, XXI, or a combination thereof.—

In the present invention, the exosomes are modified by physical methods to encapsulate a cargo. Physical methods can be passive diffusion, sonication, saponication, heating, emulsification, freezing and thawing, use of solvents, or a combination thereof. Encapsulation methods are provided in the examples herein.

In one embodiment, the encapsulation method is passive diffusion. The cargo molecule is simply incubated with the exosomes, and the cargo molecule diffuses into the exosomes along the concentration gradient. A cargo molecule and exosome can be incubated in phosphate-buffered saline at 22° C. for about 5, 10, 15, 20, 25, or 30 min.

In one embodiment, the encapsulation method is sonication. Exosomes from donor cells are mixed with a cargo and subsequently sonicated by using a homogenizer probe. The mechanical shear force from the sonicator probe compromises the membrane integrity of the exosomes and allows the cargo to diffuse into the exosomes during this membrane deformation. The membrane integrity of the exosomes has been found to be restored within an hour when the exosomes are incubated at 37° C. Cargo molecules are not only encapsulated inside the exosomes but also attached to the outer layer of the membrane.

In one embodiment, the encapsulation method is extrusion. Exosomes from donor cells are mixed with a cargo, and the mixture is loaded into a syringe-based lipid extruder with 100-400 nm porous membranes under a controlled temperature. During the extrusion, the exosome membrane is disrupted and vigorously mixed with the cargo.

In one embodiment, the encapsulation method is freeze/thawing. The exosomes are incubated with the cargo at room temperature followed by rapid freezing at −80° C. or in liquid nitrogen, and then thawing at room temperature. The process is repeated at least 3 cycles to ensure encapsulation.

In one embodiment, the encapsulation method uses a membrane permeabilizer, such as saponin. Saponin is a surfactant molecule that can form complexes with cholesterol in cell membranes and generate pores, thus leading to an increase in membrane permeabilization.

Various methods of encapsulations as described herein provide exosomes in which the lumen of the exosomes is filled with a cargo in varying percentages. For example, 1%, 2%, 3%, 5%, 7%, 10%, 15%, 20%, 25%, 30%, 35%, 40%, 45%, 50%, 55%, 60%, 65%, 70%, 75%, 80%, 85% or 90% of the lumen can be filled with a cargo. The loading efficiency depends on the hydrophobicity of the cargo molecules.

The engineered exosomes of the present invention carry one or more cargo molecules. The cargo molecules, as used herein, is a macromolecule or a compound not naturally found in the exosome. The cargo molecule can be a protein, a compound with known pharmaceutical effect on human, or a combination thereof.

As a cargo molecule, a compound with a known pharmaceutical effect on human can be codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, salts thereof, complexes thereof, or mixtures of any of the foregoing.

As a cargo molecule, an agonist of a protein useful for the present invention is an activator of Col17A1 including, but is not limited to Y27632 or apocynin. In one embodiment, keratinocytes are stimulated with Y27632 or apocynin.

The engineered exosomes described herein can carry one or more protein molecules. The protein molecules include, but are not limited to, Alix, Contactin-2/TAG1, BLBP, CAD 65, CALB 1, CD-90, CD171, CD3, CD45, CD61, CD63, CD68, CD81, CD 171, CD9, CD1, Choline transporter, Contactin-2/TAG1, DARPP-32, diverse neuron or astrocyte adhesive proteins, Dopamine Transporter (DAT), Doublecortin, EMR1, GABA Transporters 1-3, GDNF, GLAST (EAAT) , Glutamate Transporter, HES-1, Intemexin a, KA1 analogs, Laminin-1, Lhxl/5, LI NAM, LRP6 (excitatory), microglial CD18/11, MSR1, NAP 22, NCAM, Nestin, NeuN, NeuN, Notch 1, Parkin, Pcp2, RC2, RGS8, S100, SCIP analogs, Semaphorin ligands, Serotonin Transporter (SERT), Sox2, Synapsin I, TorsinA, Transferrin, TSG101, Tyro 3, VAMP, Vesicular Acetylcholine Transporter, Vesicular GABA Transporter, Vesicular Glutamate T 1-3, Zenon, Collagen type II, Desmoglein 1, Desmoglein 3, Alpha-enolase, Titin, Laminin gamma 1, Vinculin, Galectin 1, Galectin 3, Galectin 3, Heat shock protin 60, Tissue transglutaminase 2, Myelin basic protein or a combination thereof.

The engineered exosomes described herein can carry one or more microRNAs (miRNA). The miRNA includes, but is not limited to, miRNA9, miRNA335, miRNA153, miRNA21, miRNA210, miRNA34a, miRNA451, miRNA874, miRNA 124, miRNA125, miRNA 132, miRNA 134, miRNA138, miRNA106, miRNA128, miRNA 140, miRNA 146, miRNA 148, miRNA15, miRNA181, miRNA193, miRNA212, miRNA27, miRNA320, miRNA381, miRNA431, miRNA432, miRNA484, miRNA539, miRNA652, miRNA7, miRNA93, miRNA95, miRNA133, miRNA433, miRNA542, or a combination thereof. In some embodiments, exosomes derived from NSCs of the present invention carry miRNAs. These miRNAs include, but are not limited to, Homo sapiens (hsa)-miR-1246, hsa-miR-4488, hsa-miR-4508, hsa-miR-4492, and hsa-miR-4516. In one embodiment, exosomes described herein contain sufficient amount of miRNAs that could exert a biological effect in recipient cells. In one embodiment, said biological effect is regeneration, driven by miR-1246. In another embodiment, said biological effect is skeletal muscle regeneration driven by miR-4488.

The engineered exosomes described herein are less than 150 nm in diameter. For example, the exosome is about 10 nm, 20 nm, 30 nm, 40 nm, 50 nm, 60 nm, 70 nm, 80 nm, 90 nm, 100 nm, 110 nm, 120 nm, 130 nm, 140 nm, or 150 nm.

The engineered exosomes described herein comprise macromolecules encapsulated in lipid bilayer. The lipid bilayer can be about 5 nm thick, less than 5 nm or more than 5 nm thick. The lipid membrane bilayer surrounds a hydrophilic lumen in which various cargo molecules can be stored. The composition of the bilayer includes, but is not limited to, ceramide, cholesterol, sphingolipids, phosphoglycerides with long and saturated fatty-acyl chains, polysaccharide such as mannose, polylactosamine, alpha-2,6 sialic acid, N-linked glycans, or a combination thereof.

Exosomes engineered as described above are isolated from exosome-secreting cells. Described herein are methods to isolate the engineered exosomes, and then sort the exosomes according to their physical properties. In one embodiment, the physical property is the diameter of the exosomes.

Methods useful for separating the engineered exosomes include, but are not limited to, ultracentrifugation, ultrafiltration, immunoisolations, precipitation, microfluidics-based isolation, or a combination thereof. Ultracentrifugation as used herein is a centrifugation process optimized for generating exceptionally high centrifugal forces up to 1,000,000×g. Ultracentrifugation includes differential centrifugation, isopycnic ultracentrifugation, or moving-zone ultracentrifugation. Ultrafiltration as used herein is a size-based separation including the use of nanomembrane concentrators, syringe filter-based fractionation, sequential filtration, or size exclusion chromatography (SEC). Immunoisolation as used herein utilizes high-affinity antibodies conjugated to a solid surface such as glass or silica or magnetic particles. Precipitation as used herein relies on altering the solubility or dispersibility of exosomes in water-excluding polymers such as polyethylene glycol (PEG). Microfluidics-based isolation as used herein utilizes fabricated microfluidic devices employing various physical and chemical characteristics of exosomes including size, density, immunoaffinity, acoustic, electrophoretic, electromagnetic properties, or a combinations thereof. Methods of isolating and sorting the engineered exosomes are provided in the examples herein.

The engineered exosomes of the present invention release its cargo to recipient cells. The release is mediated by the fusion of the engineered exosomes with the recipient cells. The fusion can be non-specific wherein the engineered exosomes fuse with any type of cells, or cell-type specific wherein the engineered exosomes fuse with a particular type of cells. In one embodiment, the engineered exosomes release its cargo by fusing with human fibroblasts and peripheral neuron. In another embodiment, the engineered exosomes release its cargo by fusing with either human fibroblasts or peripheral neuron. In another embodiment, the recipient cells of the engineered exosomes can be epithelial cells, muscle cells, adipocytes, endothelial cells, or chondrocytes.

The cell-type specific tropism toward recipient cells can result in delivering substantially all of the cargo to only one type of cells, or in delivering about 5%, 10%, 20%, 30%, 40%, 50%, 60%, or 70% to one type of cells, and the rest are either undelivered or delivered to another cell type.

The cell-type specific tropism of the engineered exosome can be influenced by the cell type of the exosome-secreting cells. It can also be influenced by the molecules on the surface of an exosome. The surface molecules can be a naturally occurring molecules or artificially expressed molecules.

Exosomes of neuronal origins are useful for the present invention, as these exosomes are known to deliver substantially all of its cargo to neuronal cells. As a long-distance, cell-type specific cell-to-cell communication modality, exosomes budding off from neuronal cells in one location travel to neuronal cells in another location, and deliver its cargo by selectively fusing itself to neuronal cells. Molecules such as Synapsin I is correlated with cell-type specific communications via exosome. Synapsin I is found on the surface of exosomes released from neuronal cells, including glial cells, but not in non-neuronal cells. In one embodiment, the engineered exosomes of the present invention has Synapsin I on the surface of the exosome. In one embodiment, Synapsin I-containing exosomes are derived from glial cells. In another embodiment, Synapsin I-containing exosomes are derived from cultured cortical astrocytes.

The engineered exosomes described herein are designed to reach epidermis or dermis when applied to human skin. In one embodiment, the exosomes are selected by one or more size exclusion methods described herein to be about 100 nm or less in diameter. In one embodiment, the exosomes are isolated from human umbilical cord blood derived mesenchymal stem cells, glial cells, iPSCs or NSCs wherein said exosomes are less than or equal to 100 nm in diameter.

The engineered exosomes described herein are delivered to recipient cells via permeation through the skin. The permeation can be driven by passive diffusion, or pre-treating the skin for permeation. Passive diffusion is facilitated by selecting exosomes according to their size, measured in diameter, and adding exosomes of certain size in a hydrophilic solution, such as phosphate-buffered saline or Hanks balanced salt solution, and directly applying the solution to the skin. Non-limiting examples of exosome sizes useful for passive permeation are about 10 nm, 20 nm, 30 nm, 40 nm, 50 nm, 60 nm, 70 nm, 80 nm, 90 nm, 100 nm, 110 nm, 120 nm, 130 nm, 140 nm, or 150 nm.

In some embodiments, the skin is pre-treated with surfactants prior to applying exosome-containing solution of the present invention. Non-limiting examples of surfactants include sodium lauryl sulfate, ammonium lauryl sulfate, disodum lauryl sulfosuccinate, cocoamphocarboxyglycinate, cocoamidopropyl betaine, alpha-olefin sulfonate, or a combination thereof.

In some embodiments, the skin is pre-treated by physical means. These physical methods are known to those of ordinary skill in the art. Non-limiting examples of physical pre-treatment include cleaning, derma abrasion, exfoliation, chemical peel, laser resurfacing, hair removal, massaging, warming, micro-needling, laser peel, or a combination thereof.

The passive nature of exosomes fusing to the plasma membranes of the recipient cells can result in delayed release of its cargo. In some embodiments, the engineered exosomes described herein release only a fraction of its total cargo within the first month of the application. Non-limiting examples of the fraction can be 1%, 2%, 4%, 8%, 16%, 32%, 50%, 70%, or 90% of the total cargo.

In some embodiments, the formulations of the present invention comprise liquid containing free cargo molecules and engineered exosomes containing cargo molecules. As used herein, free form cargo molecules refer to cargo molecules not encapsulated within the exosomes. These formulations are useful for generating a biphasic delivery of cargo molecules wherein the two forms, i.e., the free form and the encapsulated form, have different rates of release. Non-limiting examples of the amount of the free form in the formulation can be 10%, 20%, 30%, 40%, 60% or 70%.

In some embodiments, the release of the cargo is biphasic wherein certain percentage of the cargo is rapidly released upon application of the formulation (“first phase”), and the rest of the cargo is released slowly over a long period of time (“second phase”). Non-limiting examples of the rapid release include about 10%, 20%, 30%, 40%, 60% or 70% of the cargo being released within the first phase. Non-limiting examples of the delayed release over a long period of time include releasing about 10%, 20%, 30%, 40%, 50%, 60%, or 70% of the cargo over about 3 months, 4 months, 5 months, 6 months, 7 months, 8 months, 9 months, 10 months, 11 months, or 12 months.

Compositions and methods described herein utilize differentiated human cells. The differentiated cells can repair and/or replace damaged tissue by being incorporated into the recipient tissue at the site of injury; or by replacing non-functional or damaged cells at the site of injury; or they can serve as recipient cells of the engineered exosomes described herein.

Any type of differentiated cells can be used in the present invention. Differentiated cells of the present invention can be obtained from tissue. For example, differentiated human cells can be derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, induced pluripotent stem cells, or precursor cells of the human body. Methods of establishing primary culture of differentiated cells are known in the art, and are described in the examples herein.

Differentiated human cells can also be derived from mesenchymal stem cells. For example, MSCs derived from placenta umbilical cord blood, adipose tissue, adult muscle, corneal stroma, or the dental pulp can be differentiated into osteoblasts, adipocytes, or chondrocytes. Hematopoietic stem cells differentiated into myeloid cells and lymphoid cells. Methods of deriving a particular type of differentiated cells from the mesenchymal stem cells are also known in the art, and are provided in the examples herein.

Differentiated cells of the present invention can be immune-privileged. Immune-privileged as used herein means the introduction of antigen is tolerated by the body's immune system, and does not elicit a robust inflammatory immune response resulting in the immediate rejection of the differentiated cells. Consequently, immune-privileged, differentiated cells of the present invention survive for extended periods of time without rejection.

Several methods to render the differentiated cells as immune-privileged are known in the art. Differentiated cells derived from MSCs are known to be immune-privileged, eliciting low to none immune rejection in the host. For example, in an experimental setting, osteogenic cells differentiated from MSCs did not express the MHC class II molecule, and were incapable of inducing allogeneic lymphocyte proliferation. In one embodiment, differentiated cells of the present invention are derived from MSCs. Primary cells cultured for extended periods of time are known to become immune-privileged. For example, cultured keratinocyte grafts are recognized by the host, but not rejected by CD8+T cells. In one embodiment, differentiated cells of the present invention is cultured for extended periods of time to render the cells immune-privileged. Non-limiting examples of the periods of time can be about 2 days, 4, days, 6 days, 8 days, 10 days, 12 days, 14 days, 21 days, 28 days, 2 months, 4 months or 6 months. Primary cells can also be genetically modified to minimize its immunogenicity. For example, targeted disruption of b2-microglobulin gene minimized the immunogenicity of human embryonic stem cells. In one embodiment, the differentiated cells of the present invention is genetically modified to render the cells immune-privileged.

Differentiated cells of the present invention can be immunogenic. These immunogenic differentiated cells of the present invention can be used in formulations suitable for administering in immune-privileged sites in the body. Non-limiting examples of immune-privileged sites include the eyes, the placenta, the testicles, the articular cartilage, the central nervous system, or the hair follicles.

Differentiated cells of the present invention can be autologous cells from the host. These autologous differentiated cells are derived from the tissues of the host, formulated with engineered exosomes and adhesive scaffold material described herein, and returned back to the host.

Varying amounts of the differentiated cells of the present invention can be used to prepare the compositions of the present invention. The precise amounts will depend upon a variety of factors, including other material of the composition selected for administration, whether the administration is in single or multiple doses, and individual parameters including age, physical condition, size, weight, and the stage of the disease. These factors are well known to those of ordinary skill in the art and can be addressed with no more than routine experimentation. Non-limiting examples of the amount of differentiated cells include 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

Compositions and methods described herein provide for adhesive scaffold material. “Adhesive” as used herein means material that can trap, bond to, harbor, hold, or retain differentiated cells, engineered exosomes, macromolecules or chemical compound described herein, or a combination thereof.

The scaffold material of the present invention functions as means to localize the delivery of the engineered exosomes and differentiated cells at the site of administration, as opposed to delivering the same to an unintended, wider area. Optionally, the scaffold material of the present invention functions as a delayed release means than that of the same composition, but without the scaffold material described herein. In a delayed release formulation, time for reaching the release of 100% of the cargo and differentiated cells to the site of injury can be 1.1 folds, 1.2 folds, 1.3 folds, 1.5 folds, 2 folds, 3 folds, 5 folds, 7 folds, 10 folds, 25 folds, 50 folds, 75 folds, 100 folds, 500 folds, or 1,000 folds delayed than that of the same composition without the scaffold material described herein. By way of an example, where releasing 100% of the cargo and differentiated cells may take 24 hours, the release of the same formulated with the adhesive scaffold material described herein can take 26.4 hours, 28.8 hours, 31.2 hours, 36 hours, 48 hours, 72 hours, 120 hours, 168 hours, 240 hours, 600 hours, 1200 hours, 1800 hours, 2400 hours, 12000 hours, or 24000 hours. By way of another example, where releasing 100% of the cargo and differentiated cells may take 30 minutes, the release of the same formulated with the adhesive scaffold material described herein can take 33 min, 36 min, 39 min, 45 min, 60 min, 90 min, 150 min, 210 min, 300 min, 750 min, 1500 min, 2250 min, 3000 min, 15000 min, or 30000 min.

The presence of the scaffold material can improve the delivery to the intended site of injury by slowing down the diffusion of the exosomes, cargo molecules, either encapsulated or in free form, and the differentiated cells from the site of application. Cells and exosomes formulated in a solution can quickly disperse while cells and exosomes formulated in an adhesive scaffold material can stay in the area of application, depending on the physical properties of the scaffold material.

In the present invention, suitable adhesive scaffold material include, but are not limited to, silk fibroin, cell-derived extracellular matrix (ECM), decellularized matrices, collagen matrix, or buffy coat.

Silk fibroin as used herein include, but is not limited to silk obtained from silkworms, spiders, silks genetically engineered cells, transgenic plants and animals, cultured cells, native silk, cells transfected with cloned full or partial sequences of native silk genes.

As used herein, the term “fibroin” is insoluble protein present in silk. The fibroin is obtained from a solution containing a dissolved silkworm silk or spider silk. The silkworm silk protein is obtained, for example, from Bombyx mori, and the spider silk is obtained from Nephila clawpes. In the alternative, the silk proteins suitable for use in the present invention can be obtained from a solution containing a genetically engineered silk, such as from bacteria, yeast, mammalian cells, transgenic animals or transgenic plants.

The silk fibroin as used herein may be mixed with additives that include, but are not limited to, one or more biodegradable polymers such as collagens, polylactic acid or its copolymers, polyglycolic acid or its copolymers, polyanhydrides, elastin, glycosaminoclycans, olyethylene, polystyrene, polymethylmethylcryalte, polyethylene oxide, polyurethanes, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, and hyaluronic acid.

Methods of producing silk polymer solution are provided in the examples herein. Salts useful for the solubilizing silk include, but are not limited to, lithium bromide, lithium thiocyanate, calcium nitrate or other chemical capable of solubilizing silk. Solvents useful for processing silk polymer include, but are not limited to, hexa-fluoro-iso-propanyl (HFIP), N-methyl morpholine N-oxide and calcium nitrate-methanol.

To form a scaffold, the silk polymer/solvent solution of the present invention is placed into a form, or mold, containing water-soluble particles, or porogens, that are insoluble in organic solvents. Alternatively, the porogens are mixed with the silk polymer solution prior to placement in the mold. The diameter of the particles are preferably between about 50-1000 microns. Examples of water-soluble porogens useful according to the present invention include, NaCl, alkali metals, alkali earth metal halides, phosphates, and sulfates, sugar crystals, water-soluble microspheres, polysaccharides and protein microspheres. The solvent is consequently removed using, for example, sublimation or evaporation. The polymer solution is treated with an effective amount of alcohol to induce β-sheet structure and insolubility in aqueous solution. The composite or polymer is immersed in water or other solvent in which the particles, or porogens are soluble and polymer is insoluble, to remove the particles, resulting in a porous three-dimensional structure.

For the fabrication of insoluble scaffold suitable for, e.g., bone regeneration, regenerated silk fibroin is immersed in an alcohol or other suitable agent to obtain the β-sheet structure. Therefore, prior to submersion into aqueous solutions the silk scaffolds are first soaked in a β-sheet structure inducing agent, such as alcohol to induce the phase transition to β-sheet structure. The type of a β-sheet structure inducing agent can be used to generate scaffolds with different properties. When methanol and propanol are used to induce β-sheet structure, the resulting scaffolds are stronger but more brittle.

ECM scaffold as used herein can be prepared from cells. For example, chondrocytes are extracted from animal cartilage, and then culture for 3-4 days to form a thin layers of chondrocyte/extracellular matrix membrane. The cell layers having the ECM membrane is carefully separated, and transformed to a pellet-shape through centrifugation. The pellet-shape is then cultured for about 3 weeks for the growth of new cartilage tissue. The newly grown ECM layers are harvested, and treated with 3 freeze-thaw cycles. The ECM layers are decellularized with proteinase such as 0.05% trypsin; detergent such as SDS, Triton-X, or deoxycholate; ultrasound; DNase, or a combination thereof. The decellularized ECM is then lyophilized, and ground into fine powder. To construct a scaffold of any shape, the powdered ECM can be dissolved in an acid solution, and then formed into a desirable form using a mold, followed by further modification using chemical cross-linkers.

Another way to prepare the ECM is to use human fibroblast. Human fibroblast derived extracellular matrix can be obtained from cultured human lung fibroblasts via a mild decellularization process. A method of preparing ECM from human lung fibroblasts, and combining the ECM with differentiated cells and exosomes is provided in the examples herein. Heparin is a major component of the extracellular matrix. To increase the biocompatibility of the scaffold, the lung fibroblast derived ECM can be conjugated with heparin via N-(3-Dimethylaminopropyl)-N′-ethylcarbodiimide hydrochloride (EDC) chemistry. Another major component of the extracellular matrix is collagen. Collagen can be added to the lung fibroblast derived ECM by mixing purified type I collagen with the heparin-conjugated ECM.

Buffy coat is a thin yellowish layer, which is the middle layer, in the centrifuge tube when a sample of blood is centrifuged. Top layer is plasma, and the bottom layer contains erythrocytes. Buffy coat contains leukocytes and platelets. Buffy coat possesses the physical property of being adhering to solid substrates, such as bone and cartilage, as well as semi-solid substrates such as muscle and other tissue, and thus may be used to provide a type of temporary “glue” to hold the differentiated cells and engineered exosomes in place at the site of administration so that localized delivery is achieved.

In one embodiment, buffy coat is mixed with differentiated cells and engineered exosomes of the present invention, and then administered to a site of injury.

There are no limits as to how much of the purified buffy coat may be used to formulate the compositions of the present invention, so long as the amount is effective to substantially increase the localized delivery of the composition at the site of administration. Thus, the ratio of amount of buffy coat to a mixture of differentiated cells and exosomes can be in the range of about 1-5 to 1 by volume percent of the injectable composition. Typically, the range can include about 1-3 to 1. In particular, the range can be about 1 to 1 in terms of the volume of buffy coat and a mixture of differentiated cells and exosomes.

Decellularized matrices as used herein means biological scaffold material sourced from tissue by removing cells and DNA from the tissue. Decellularized matrices can be prepared from living body tissue, cadaver, amnion, or umbilical cord. Several methods of decellularizing are known in the art. These methods include the use of surfactant, digestive enzymes, oxidizing agent, using high-hydrostatic pressure, freeze-thaw, hypertonic electrolytic solution, or a combination thereof.

In one embodiment, decellularized matrices are prepared by a high hydrostatic pressure method. The source tissue is washed with washing liquid. The washing liquid contains surfactant. The surfactant can be 0.01% to 2% of the liquid by weight. The washing is performed using a hydrostatic pressure of 50 MPa to 1200 MPa, at a temperature between 0° C. to 45° C. for 5 minutes to 60 minutes. After washing with the source tissue, the tissue is treated with nuclease.

Surfactants include anionic surfactants such as fatty acid soap, alkoxy carboxylate, polyoxyethylene alkoxy carboxylate, alkyl sulfonate, alkylbenzene sulfonate, alkyl sulfuric acid ester salts, polyoxyethylene alkyl sulfuric acid ester salts, alkyl phosphoric acid ester salts, α-sulfofatty acid ester salt, N-acylglutamate, acyl-N-methyltaurine salt, N-alkylsarcosine salt, cholate, or deoxycholate. Cationic surfactants include alkyldimethylamine, alkyldiethanolamine, alkyltrimethylammonium salts, dialkyldimethylammonium salts, alkylpyridinium salt, or alkylbenzyldimethylammonium salt. Amphoteric surfactants include alkyldimethylamine oxide, alkylcarboxybetaine, alkylamidepropylbetaine, alkylamidepropylsulfobetaine, alkylimidazonliumbetaine, 3-[4(3-cholamidopropyl)dimethylammonio]-2-hydroxy-1-propanesulfonate (CHAPSO), or 3-[(3-cholamidopropyl)dimethylammonio]-1-propanesulfonate (CHAPS). Non-ionic surfactants include glycerin fatty acid ester, sorbitan fatty acid ester, sucrose fatty acid ester, trehalose fatty acid ester, polyethylene glycol fatty acid ester, polyoxyethylene glycerin fatty acid ester, polyoxyethylene sorbitan fatty acid ester, polyoxyethylene alkyl ether, polyoxyethylene alkyl phenyl ether, polyoxyethylene polyoxypropylene ether, alkyl(poly)glycerin ether, fatty acid alkanolamide, polyoxyethylene fatty acid alkanolamide, alkyl(poly)glycoside, alkyl maltoside, alkylthio glucoside, alkyl maltopyranoside, alkanoyl-N-methyl-D-glucosamine, N,N-bis(3-D-gluconamidepropyl)cholamide (BIGCHAP), or N,N-bis(3-D-gluconamidepropyl)deoxycholamide (deoxy-BIGCHAP).

In addition to surfactant, the washing liquid can also contain water, physiological saline, propylene glycol or an aqueous solution thereof, glycerin or an aqueous solution thereof, aqueous saccharide, and the like. Buffer solutions include acetate buffer solutions, phosphate buffer solutions, citrate buffer solutions, borate buffer solutions, tartrate buffer solutions, Tris buffer solutions, HEPES buffer solutions, IVIES buffer solutions, and the like. Saccharides in aqueous saccharide include erythrose, xylose, arabinose, allose, talose, glucose, mannose, galactose, erythritol, xylitol, mannitol, sorbitol, galactitol, sucrose, lactose, maltose, trehalose, dextran, alginic acid, hyaluronic acid, and the like.

After the wash, the source tissue can be treated further with nuclease, an organic solvent, or a chelating agent. Nuclease can improve an efficiency of removing a nucleic-acid component from a living body tissue to which a hydrostatic pressure has been applied. An organic solvent can improve an efficiency of removing lipid. A chelating agent can prevent calcification by inactivating calcium ions and magnesium ions in a decellularized tissue when the particulate decellularized tissue according to the present invention is applied to an affected area. As an organic solvent, a water-soluble organic solvent is preferred in view of a high removal efficiency of lipid, and ethanol, isopropanol, acetone, or dimethyl sulfoxide are preferred. Chelating agents include iminocarboxylic-acid based chelating agents and salts thereof, such as ethylenediaminetetraacetic acid (EDTA), nitrilotriacetic acid (NTA), diethyl enetriaminepentaacetic acid (DTPA), hydroxyethylethylenediaminetriacetic acid (HEDTA), triethylenetetraminehexaacetic acid (TTHA), 1,3-propanediaminetetraacetic acid (PDTA), 1,3-diamino-2-hydroxypropanetetraacetic acid (DPTA-OH), hydroxyethyliminodiacetic acid (HIDA), dihydroxyethylglycine (DHEG), glycoletherdiaminetetraacetic acid (GEDTA), dicarboxymethylglutamic acid (CMGA), 3-hydroxy-2,2′-iminodisuccinic acid (HIDA), dicarboxymethylaspartic acid (ASDA); or hydroxycarboxylic-acid based chelating agents and salts thereof, such as citric acid, tartaric acid, malic acid, or lactic acid. Salts of these chelating agents include sodium salts or potassium salts.

Collagen matrix as used herein means a mashed network of collagen fibers. Collagen matrix can contain Type I, II, or III collagens, or a mixture thereof. Collagen can be harvested from an animal source, including human, bovine, porcine, or fish. Harvested collagen are washed extensively to remove cells, especially blood cells, cell debris, and/or DNA. After the wash, the collagen raw material is dissolved in water or a buffered solution, and incubated for 1 day, 2 days, 3 days, 4 days, 5 days, 6 days, or 7 days, to be expanded in volume, and form a porous mesh. The dissolved collagen raw material is poured into a tray, and freeze-dried to form a sheet. Optionally, the freeze-dried collagen sheet can be ground to form powder. As a freeze-dried scaffold material, the collagen matrix can be 99% dry, 98% dry, 97% dry, 96% dry, or 95% dry, containing up to 1%, 2%, 3%, 4%, or 5% water.

The engineered exosomes described herein are formulated into cosmetic composition utilizing media suitable for applying to human skin. In some embodiments, the medium contains emollients. An emollient as used herein is esterification product of an aliphatic alcohol having at least 25 carbon atoms and an unsaturated aliphatic fatty acid having at least 16 carbon atoms. Non-limiting examples of emollients are Cerotyl linoleate, Diisoestearyl dimerate, Diisopropyl dimerate, Stearyl oleate, Stearyl linoleate, Cerotyl oleate, Melissyl oleate, Melissyl linoleate, or Cerotyl eicosapentanoate.

In some embodiments, the medium is a buffered solution, such as phosphate-buffered saline, Hanks balance salt solution, or saline. Optionally, the solution may contain cosmetically acceptable preservatives.

In some embodiments, the engineered exosomes described herein are formulated as a cosmetic product, such as a balm, spray, gel, cream, serum, or a toner. Typical ingredients forming these various cosmetic base are known in the art.

In some embodiment, the cosmetic formulation used herein is compatible with an ingredient covered with lipid membrane, such as exosome, without negatively affecting the integrity of the lipid membrane structure. Non-limiting examples of compatible cosmetic ingredient include water, disodium EDTA, soldium hyaluronate, glycerin, palmitoyl tripeptide, dextran, caproyl tetrapeptide, xanthan gum, cetostearyl alcohol, shea butter, jojoba oil, coconut oil, cetearyl ethylhexanoate, squalene, carbomer, pheoxyethanol, ethylhexylglcerin, NaOH, corundum powder, Asparagopsis armata extract, collagen, magnesium stearate, cellulose, lactose, mannitol, methyl cellulose, or a combination thereof.

In some embodiments, the formulations containing the engineered exosomes described herein are housed in a device capable of delivering the exosomes to the skin. Non-limiting examples of the mechanical devices include a roller, a ball-pointed liquid container, a liquid dispenser, a sprayer, a spatula, a dropper, a hand-operated pump, a tube, or a combination thereof. A particular design of the device and specification thereof depends on the amount, frequency, and the area of the delivery of the formulations. A variety of designs capable of holding and delivering formulation containing liposomes are known in the art, and these device designs are compatible with the exosome-containing formulations of the present invention.

Compositions and methods described herein are formulated as a pharmaceutical composition utilizing pharmaceutically acceptable excipients. Excipients for the present invention provides stability to the macromolecules or adhesive scaffold materials described herein, viability of the human differentiated cells described herein, and are acceptable as an injectable fit for human use. Excipients useful for the present invention include, but are not limited to, cell culture medium, such as alpha medium without phenol red, buffered solutions, such as phosphate buffered saline or hanks balanced salt solution, Ringer's solution, dimethyl sulfoxide, chelators, such as EDTA, glycerol, polyethylene glycol, sorbitol, mannitol, water, ions such as sodium, potassium, chloride, amino acids, citric acid, lactic acid, or a combination thereof.

Each excipient or carrier must be acceptable in the sense of being compatible with the other ingredients of the formulation and not injurious to the subject. These acceptable amounts for pharmaceutical carriers depend upon a variety of factors, including the material selected for administration, whether the administration is in single or multiple doses, and individual parameters including age, physical condition, size, weight, and the stage of the disease. These factors are well known to those of ordinary skill in the art and can be addressed with no more than routine experimentation.

The formulations of the invention are administered in effective amounts. An effective amount is that amount of an agent that alone stimulates the desired outcome. The absolute amount will depend upon a variety of factors, including the material selected for administration, whether the administration is in single or multiple doses, and individual parameters including age, physical condition, size, weight, and the stage of the disease. These factors are well known to those of ordinary skill in the art and can be addressed with no more than routine experimentation.

The formulations may be prepared with pulmonary surfactants. A pulmonary surfactant is a lipoprotein mixture useful in keeping lung airways open (e.g., by preventing adhesion of alveolar walls to each other). Pulmonary surfactants may be comprised of phospholipids such as dipalmitoylphosphatidylcholine (DPPC), phosphotidylcholine (PC), phosphotidylglycerol (PG); cholesterol; and proteins such as SP-A, B, C and D. Pulmonary surfactants may be derived from naturally occurring sources such as bovine or porcine lung tissue. Examples include Alveofact™ (from cow lung lavage), Curosurf™ (from minced pig lung), Infasurf™ (from calf lung lavage), and Survanta™ (from minced cow lung, with additional components including DPPC, palmitic acid, and tripalmitin). Pulmonary surfactants may also be synthetic. Examples include Exosurf™ (comprised of DPPC with hexadecanol and tyloxapol), Pumactant™ or Artificial Lung Expanding Compound (ALEC) (comprised of DPPC and PG), KL-4 (comprised of DPPC, palmitoyl-oleoyl phosphatidylglyercol, palmitic acid, and synthetic peptide that mimics SP-B), Venticute™ (comprised of DPPC, PG, palmitic acid, and recombinant SP-C). Pulmonary surfactants may be obtained from commercial suppliers.

The engineered exosomes of the present invention can be formulated as low or high dosage forms. Low dosage forms may range from, without limitation, 1-50 micrograms per kilogram, while high dosage forms may range from, without limitation, 51-1000 micrograms per kilogram. In some embodiments, the engineered exosomes of the present invention are formulated as 3 mg, 4 mg, 5 mg, 6 mg, 7 mg, 8 mg, 9 mg, 10 mg, 50 mg, or 80 mg per package.

The compositions described herein can be administered repeatedly, including two, three, four, five or more administrations. In some instances, the compositions may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the purpose of the administration. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ.

Described herein is a method of selecting compositions suitable for carrying engineered exosomes, differentiated cells, and adhesive scaffold material to a site of injury.

To perform the selection, exosome-secreting cells are cultured as described above. The exosome-secreting cells are then genetically modified to express one or more macromolecules described herein. Alternatively, unmodified exosomes are harvested from the exosome-secreting cells, and the exosomes are loaded with one or more macromolecules, chemicals, or a combination thereof described herein. The isolated exosomes are mixed with differentiated human cells, adhesive scaffold material, and pharmaceutically acceptable excipients described herein. The formulation is then administered to a human subject at a site of injury to examine the clinical responses of a human subject. Formulations eliciting desired therapeutic effects are recorded.

As used herein, a “site” or a “site of injury” means tissue injury in human including, but is not limited to, injuries caused by wear and tear, deterioration, degradation, degeneration, trauma, loss of cells or tissues, loss of function, idiopathic, genetic, metabolic, environmental exposure, aging, or a combination thereof.

In one embodiment, the site of injury is connective tissue suffering from osteoarthritis. In one embodiment, the site of injury is synovium, meniscus, or defective cartilage.

As used herein, a clinical response means a measurable response including, but is not limited to, the use of diagnostic devices, patient reported outcome, questionnaire, survey, physical examination, observations slumend by a healthcare practitioner, or a combination thereof.

In one embodiment, the clinical response is measuring the knee function assessed by international knee documentation committee (IKDC) scoring. In another embodiment, the clinical response is assessing pain in the knee by visual analog scale (VAS), Lysholm knee scoring, Knee injury and osteoarthritis outcome scoring (KOOS), magnetic resonance imaging, or a combination thereof.

As used herein, a desired therapeutic effect refers to an improvement in clinical responses. An improvement can be an increase or a decrease in a scoring method, recorded by a unit of measure employed in a particular scoring method. An improvement can be expressed as a percentage, as in 1%, 2%, 3%, 5%, 7%, 10%, 20%, 30%, 40%, 50%, 70%. 85%, 90%, 100%, 200%, 500%, 750%, 1,000% improved over non-treated condition.

### EXAMPLE

### Isolating Exosome-Secreting Neuronal Cells

Tissues containing primary cortical neurons are harvested from the brain, and washed with phosphate-buffered saline containing 1.5% glucose. The tissue is dissociated by passing through a 21 gauge needle. The cells are then separated from tissue debris by allowing the tissue debris to settle in a tube, and by transferring the supernatant to a new tube. To collect the cells, the supernatant is spun down for 5 min at 134×g. The collected cells are resuspended in a culture medium, such as commercially available Neurobasal media (Invitrogen™), supplemented with 2% B27 (Invitrogen™), 2 mM glutamine, 1.5% glucose, 100 ug/ml streptomycin and 60 ug/ml penicillin, and plated at a density 0.5×10⁶ cells per well in a 24-well plate precoated with 10 ug/ml poly-D-lysine. The culture is grown at 37° C. in a humidified atmosphere of 95% air and 5% CO₂. The neuronal identity of the culture can be confirmed by beta-tubulin III staining. After 7 days, the cells can be plated onto 5 ug/ml poly-D-lysine coated 100 mm dishes at a density of 6×10⁶ cells/dish.

### Example 2

### Isolating Primary Astrocytes Secreting Exosomes

From the brain tissue, the meninges are discarded. Then the cortices are minced, which is followed by the dissociation of the by trituration through a 2 ml pipet. The dissociated tissue is incubated with trypsin to release the cells. The cells are then seeded in 10 cm plates in DMEM medium containing 10% fetal calf serum and 1% penicillin/streptomycin.

### Example 3

### Isolating NSCs from the Brain

The SVZ and DC are dissected as follows: 1) place the rostral portion of the brain so that the cut coronal surface faces upwards and focus the microscope onto a higher magnification, 2) remove and discard the septum using fine curved forceps, 3) dissect the SVZ (the thin layer of tissue surrounding the ventricle) by placing the tip of one blade of a pair of fine curved forceps in the lateral corner of the lateral ventricle immediately under the corpus callosum and the other approximately 1 mm into the tissue immediately adjacent to the ventricle, 4) press down the forceps towards the base of the dish and towards the ventral aspect of the ventricle to remove a small triangular piece of tissue, and 5) place the dissected SVZ into a Petri dish on ice. To dissect the DG, 1) place the caudal portion of the brain in the Petri dish and cut along the longitudinal fissure using a scalpel, 2) under a dissection microscope, remove the cerebellum and the diencephalon using forceps, 3) refocus the microscope so that the borders around the DG are now visible, 4) to remove the dentate gyms, insert the tip of a 27 G needle and slide along the border between the DG and Ammon's horn, and 4) using the fine forceps, free the DG from the surrounding tissue. Once SVZ is dissected, mince the tissue using a scalpel until no large pieces remain. The minced tissue is treated with 0.05% Trypsin-EDTA for 7 min at 37° C. The trypsin-digested tissue is centrifugated at 300 g for 5 min to pellet the cells. The cells are resuspended in growth medium comprising Neuralbasal media supplemented with 2% B27, 2 mM glutamine, 2 ug/ml heparin, and 50 units of penicillin/streptomycin, 20 ng/ml of epidermal growth factor, and 20 ng/ml of fibroblast growth factor. For dissected DG tissue, mince the tissue until no large pieces remain, and treat the tissue with 2.5 units/ml Papain, 1 unit/ml dispase, 250 units/ml DNase I for 20 min at 37° C. The digested tissue is then centrifuged at 130×g for 5 min to pellet the cells, and resuspended in 1 ml buffer solution ((1×HBSS, 30 mM Glucose, 2 mM HEPES (pH 7.4), 26 mM NaHCO₃), and centrifuged again. The NSCs from DG are then isolated as follows: 1) remove supernatant and resuspend the pellet in 5 ml of 20% Percoll. (To prepare 90% Percoll, add 4.5 ml of 100% Percoll to 0.5 ml of 10×PBS then further dilute this to 20% by adding 1.1 ml 90% Percoll to 3.9 ml 1×PBS), 2) centrifuge 450×g for 15 min, 3) remove the supernatant and resuspend the pellet in 10 ml buffer, 4) centrifuge at 130×g for 5 min, and 5) resuspend the pellet in 200 ul growth medium.

### Example 4

### Culturing iPSCs and Deriving NSCs from the iPSCs

iPSCs are grown on a feeder layer made of irradiated mouse embryonic fibroblasts (iMEF) or on a coated plate such as Matrigel (BD), Cellstart (Invitrogen) or Vitronectin XF (Stem Cell Technologies) as feeder-free culture. For feeder layer, about 200,000 cells are plated in a well of a 6-well plate. For passaging of the cells, mechanical methods, such as colony picking or an enzymatic method can be used. Enzymes suitable for passing iPSC colonies are Collagenase type IV diluted with DMEM-F12, or Dispase. iPSC are detached from plates using Dispase (1 mg/ml) for 30min at 37° C. with the help of a scraper. To form embryonic bodies, colonies are collected by sedimentation and resuspended in a medium comprising Knockout™ DMEM, Knockout™ SR, non-essential amino acids, penicillin/streptomycin, β-mercaptoethanol. The medium is supplemented with 10 MSB-431542 (Ascent Scientific), 1 μM dorsomorphin (Tocris), 3 μLM CHIR 99021 (Axon Medchem), which is a GSK-3 inhibitor, and 0.5 μM purmorphamine (Alexis, also known as PMA or 2-(1 -Naphthoxy)-6-(4-morpholinoanilino)-9-cyclohexylpurine), which is a Sonig Hedgehog signaling and WNT-signaling activator. 48 hours later, the medium is replaced by N2B27 medium comprising DMEM-F12 and Neurobasal medium mixed at 50:50 ratio, and supplemented with 1:200 N2 supplement (Invitrogen), 1:100 B27 supplement lacking vitamin A (Invitrogen), penicillin/streptomycin and glutamine, supplemented with 10 μM SB-431542, 1 μM dorsomorphin, 3 μM CHIR 99021 and 0.5 μM PMA. After additional 48 hours, the medium is replaced by N2B27 medium supplemented with 3 μM CHIR 99021, 0.5 μM PMA and 150 μM ascorbic acid (Sigma Aldrich), which is an antioxidant. Two days later, neural tube like structures are dissociated by pipetting, and small pieces are plated on Matrigel (BD Biosciences) coated 12-well plates in N2B27 medium supplemented with 3 μM CHIR 99021, 0.5 μM PMA and 150 μM ascorbic acid. At day 8, the medium is exchanged by N2B27 medium supplemented with 3 μM CHIR 99021, 0.5 μM PMA, 150 μM ascorbic acid and 20 ng/ml basic fibroblast growth factor 2 (bFGF2). At day 12, cells are detached using dispase and cultivated in a medium on Matrigel coated 10 cm dishes comprising DMEM HAM's F12 medium supplemented with 200 ng/ml epidermal growth factor, 200 ng/ml bFGF2, N2 supplement, B27 supplement with vitamin A, glutamine, penicillin/Streptomycin and 150 unit/ml of human leukocyte inhibitory factor (LIF).

### Example 5

### Differential Ultrafiltration of Exosomes

A mixture of live cells, dead cells and debris, culture medium, and exosomes are placed in a tube and centrifuged at 300×g for 10 min. The live cells are discarded, and the supernatant containing the rest are centrifuged at 2,000×g for 10 min. The dead cells are discarded and the remaining supernatant is centrifuged at 10,000×g for 30 min to remove cell debris. The supernatant, which contains exosomes and culture medium, is centrifuged at about 100,000-200,000×g for 70 min to pellet the exosomes. The supernatant, which contains culture medium, is discarded, and the exosome pellet is resuspended in a suitable solution, such as phosphate buffered saline, and re-centrifuged at about 100,000-200,000×g for 70 min.

### Example 6

### Isopycnic Ultracentrifugation of Exosomes

A density gradient medium encompassing the entire range of densities of solutes in a sample is loaded to a centrifuge tube. After removing the cell debris, the supernatant is placed on a density gradient, and centrifuged at about 100,000-200,000×g, which results in exosome forming a narrow band suitable for extraction. A density medium includes, but is not limited to, cesium chloride. In this method, exosomes are extracted from the density region between 1.10 and 1.21 g/ml. Once the band containing exosomes is extracted, it is subjected to a brief centrifugation at about 100,000×g to further purify the exosomes.

### Example 7

Moving-Zone Ultracentrifugation of Exosomes

A sample containing exosomes is loaded as a thin zone on top of a gradient density medium of having a lower density than that of any of the solutes. The exosomes in the sample are then separated based on their size and mass instead of density. This allows the separation of exosomes with similar densities but different sizes.

### Example 8

Using a Nanomembrane Concentrator to Isolate Exosomes

A nanomembrane concentrator has a polyethersulfone membrane and a uniform pore size of 13 nm. Such a nanomembrane concentrator provides exosomes with less than 100 nm in diameter. After brief centrifugation to remove cells and cell debris, the clear supernatant containing exosomes are added to nanomembrane concentrator, and then centrifuged at 3,000×g at 20° C. for 10-30 min. The retentate is recovered, and the membrane is washed with solubilizing buffer, and combined with the retentate to increase the yield of exosomes.

### Example 9

### Using a Syringe Filter-Based Fractionation to Isolate Exosomes

A syringe is equipped with two membranes in tandem configuration so that exosomes are captured on the lower membrane whereas larger extracellular vesicles are retained on the upper membrane when a sample is passed through the two membranes. The syringe is filled with exosomes-containing sample, and forced to pass through the filters. Exosomes, based on their sizes, are trapped between the two membranes.

### Example 10

### Sequential Filtration of Exosomes

Samples containing exosomes, cells, and cell debris are filtered using 100 nanometer filter, depleting floating cells and larger debris from the sample. The filtered sample is subjected to a second filtration using hollow fibers with 500 kDa molecular weight cut-off. The retentate is then filtered with 100 nm track-etch filter whereby exosomes are recovered from the filtrate.

### Example 11

Size-Exclusion Chromatography

A size-exclusion column having a porous stationary phase is used to sort macromolecules and particulate matters according to their sizes. The instrument comprises a liquid chromatography system with a binary pump, an auto injector, a thermostated column oven, and a UV-visible detector operated by the Class VP software (Shimadzu Corporation). The Chromatography columns are TSK Guard column SWXL, 6×40 mm, and TSK gel G4000 SWXL, 7.8×300 mm (Tosoh Corporation). Detectors, Dawn 8 (light scattering), Optilab (refractive index) and QELS (dynamic light scattering) are connected in series following the UV-visible detector. The eluent buffer is 20 mM phosphate buffer with 150 mM NaCl, pH 7.2. This buffer is filtered through a pore size of 0.1 μm and degassed for 15 min before use. The chromatography system is equilibrated at a flow rate of 0.5 ml/minute until the signal in Dawn 8 is stabilized at around 0.3 detector voltage units. The UV-visible detector is set at 220 nm and the column's temperature is equilibrated to 25 ° C. Isocratic elution mode is run for 40 min. The volume of sample containing exosomes ranges from 50 to 100 ul. The % area of the exosome peak vs. all other peaks are integrated from the UV-visible detector. The hydrodynamic radius, Rh is computed by the QELS and Dawn 8 detectors. The highest count rate (Hz) at the peak apex is taken as the Rh. Peaks of the separated components visualized at 220 nm are collected.

### Example 12

### Magnetic Separation of Exosomes

Antibodies against molecules at the surface of exosomes are utilized to capture exosomes. Anti-CD63 antibodies are conjugated with magnetic particles, added to the sample containing exosomes. The mixed sample is then placed in a tube. Upon placing the tube in a magnetic field, exosomes having CD63 on their surface are bound to the inside wall of the tube by the magnetic field. The sample is then repeated washed with phosphate buffered saline to remove unbound particulates. After the wash, the tube is detached from the magnets, and the washed exosomes are released.

### Example 13

### Selective Precipitation of Exosomes

Polyethylene glycol (PEG) is used to isolate exosomes. A sample containing exosomes is pre-cleared by passing through a Sephadex G-25 column, removing subcellular particles such as lipoproteins. The cleared sample is then incubated in a solution containing PEG with a molecular weight of 8,000 Da. After incubation at 4° C. overnight, the precipitate containing exosomes is isolated by means of either low speed centrifugation or filtration.

### Example 14

### Microfluidics Separation of Exosomes

Microfluidic devices having fabricated surfaces are designed to isolate exosomes based on their properties. Exosomes are isolated using an acoustic nanofilter, utilizing ultrasound standing waves to separate exosomes according to their size and density. An acoustic nanofilter moves larger particles faster toward a node at the end of a channel, as they experience stronger radiation forces. Alternatively, a porous silicon-based nanowire having a micropillar structure is fabricated on a surface. The micropillars are spaced in a lattice with fixed distances to trap exosomes with diameters less than 100 nm. The trapped exosomes are recovered by dissolving the porous silicon nanowires in phosphate buffered saline.

### Example 15

### Transient Expression of TGF-Beta1

To transiently express TGF-beta1 of interest, about 1.5×10⁵ cells are plated in a 35 mm tissue culture dish containing 3 mL of complete Dulbecco's Modified Eagle Media (DMEM), supplemented with 10% fetal bovine serum (FBS), 1× penicillin/streptomycin solution (Invitrogen) and 1×MEM non-essential amino acids solution (MEM) (Invitrogen, Inc), and are grown In a 37° C. incubator under 5% carbon dioxide until cells reach a density of about 5×10⁵ cells/mL (6-16 hours). A 500 uL transfection solution is prepared by adding 250 ul of OPTIMEM Reduced Serum Medium containing 15 μL of UpofectAmine 2000 (Invitrogen, Carlsbad, Calif.) incubated at room temperature for 5 minutes to 250 μL of OPTI-MEM Reduced Serum Medium containing expression plasmid carrying the cDNA of TGF-beta1. This transfection is incubated at room temperature for approximately 20 min. The complete, supplemented DMEM media is replaced with 2 mL of OPTI-MEM Reduced Serum Medium and the 500 uL transfection solution is added to the cells, and the cells are incubated In a 37° C. incubator under 5% carbon dioxide for approximately 6 to 18 hours. Transfection media is replaced with 3 mL of fresh complete, supplemented DMEM and the cells are incubated in a 37° C. incubator under 5% carbon dioxide for 48 hours.

### Example 16

### Confirming the Transient Expression of TGF-Beta1

Cells are harvest by rinsing cells once with 3.0 mL of 100 mM phosphate-buffered saline, pH 7.4 and lysing cells with a buffer containing 50 mM N-(2hydroxyethyl) piperazine-N′.(2-ethanesufonic acid) (HEPES), pH 6.8 150 mM sodium chloride, 1.5 mM magnesium chloride, 10% (v/v) glycerol, 1 mM ethylene glycol bis(j-aminoethyl ether) N,N,N,N′tetraacetic acid (EGTA), 2% (v/v) Triton-X 100 (4-octylphenol polyethoxylate) and 1×Complete protease inhibitor cocktail (Roche Applied Science). Cell samples are added to 2×LDS Sample Buffer (Invitrogen, Inc) and expression is measured by Western blot analysis.

### Example 17

### Stable Expression of TGF-Beta1

To generate a stably-integrated cell line expressing TGF-beta1, approximately 1.5×10⁵ HDFa cells are plated in a 35 mm tissue culture dish containing 3 ml of complete DMEM, supplemented with 10% FBS, 1× penicillin/streptomycin solution (invitrogen) and lx MEM nonessential amino acids solution (Invitrogen), and grown in a 37° C. incubator under 5% carbon dioxide until cells reach a density of about 5×10⁵ cells/mI (6-16 hours). A 500 ul transfection solution is prepared by adding 250 ul of OPTI-MEM Reduced Serum Medium containing 15 ul of LipofectAmine 2000 (Invitrogen) incubated at room temperature for 5 min to 250 ul of OPTI-MEM Reduced Serum Medium containing 5 ug of a plasmid containing the cDNA of TGF-beta1 and a selection marker for stable transfection. This transfection is incubated at room temperature for approximately 20 min. The complete, supplemented DMEM media is replaced with 2 mL of OPTI-MEM Reduced Serum Medium and the 500 ul transfection solution is added to the HDFa cells and the cells are incubated in a 37° C. incubator under 5% carbon dioxide for approximately 6 to 18 hours. Transfection media is replaced with 3 ml of fresh complete, supplemented DMEM and the cells are incubated in a 37° C. incubator under 5% carbon dioxide for approximately 48 hours. Media is replaced with 3 ml of fresh complete DMEM, containing 5 ug/mL blasticidin, 10% FBS, 1' penicillin/streptomycin solution (Invitrogen) and 1 x MEM non-essential amino acids solution (Invitrogen). Cells are incubated in a 37 OC incubator under 5% carbon dioxide for approximately 3-4 weeks, with old media being replaced with fresh blasticidin selective, complete, supplemented DMEM every 4 to 5 days. Once blasticidin-resistant colonies are established, resistant clones are replated to new 35 mm culture plates containing fresh complete DMEM, supplemented with approximately 5 ug/mL of blasticidin, 10% FBS, 1× penicillin/streptomycin solution (Invitrogen, Inc) and 1×MEM non-essential amino acids solution (Invitrogen), until these cells reach a density of 6 to 20×10⁵ cells/mL.

### Example 18

### Confirming the Stable Expression of TGF-Beta1

To test for expression of TGF-beta1 from HDFa cell lines that have stably-integrated a cDNA for TGF-beta1, approximately 1.5×10⁵ HDFa cells from each cell line are plated in a 35 mm tissue culture dish containing 3 mL of blasticidin selective, complete, supplemented DMEM and grown in a 37 OC incubator under 5% carbon dioxide until cells reach a density of about 5×10⁵ cells/ml (6-16 hours). Media is replaced with 3 mL of fresh blasticidin selective, complete, supplemented DMEM and cells are incubated in a 37° C. incubator under 5% carbon dioxide for 48 hours. Both media and cells are collected for expression analysis of TGF-beta1. Media is harvested by transferring the media to 15 mL snap-cap tubes and centrifuging tubes at 500×g for 5 min to remove debris. Cells are harvest by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2×LDS Sample Buffer (Invitrogen) and expression is measured by Western blot analysis using anti-TGF-beta1 antibodies. The established HDFa cell line showing the highest expression level of TGF-beta1 is selected for large-scale expression using 3 L flasks. Procedures for large-scale expression are as outlined above except the starting volume is approximately 800-1000 mL of complete DMEM and concentrations of all reagents are proportionally increased for this volume.

### Example 19

### Encapsulation by Sonication

A cargo molecule is mixed with about 10¹¹ exosomes in 1 ml phosphate-buffered saline. The mixture is incubated at 37° C. for 1 hour with shaking. The mixture is then sonicated at 20% amplitude, 6 cycles of 30 seconds on and off for three minutes with a two-minute cooling period between each cycle. After the sonication, the mixture is incubated at 37° C. for 1 hour to allow for recovery of the exosomes.

### Example 20

### Encapsulation by Extrusion

Exosomes are prepared at a concentration of 0.15 mg/ml of total protein. The cargo protein is prepared in PBS at a concentration of 0.5 mg/ml. The exosomes and the cargo protein are mixed at a ratio about 4:6, and then extruded 10 times through a commercial extruder, such as Avanti Lipids extruder, with 200 nm-pores diameter. The loaded exosomes are purified by gel-filtration chromatography with Sepharose column.

### Example 21

### Encapsulation Using a Permeabilizer

Exosomes are prepared at a concentration of 0.15 mg/ml of total protein. The cargo protein is prepared in PBS at a concentration of 0.5 mg/ml. The exosomes and the cargo protein are mixed at a ratio about 4:6, and incubated for 20 min in the presence of 0.2% saponin.

### Example 22

### Encapsulation by Transfection

1 ug of the DNA is mixed with 1 ul of the transfection reagent, and 3 ug of exosomes in 100 ul of DMEM. The solution is incubated for 3 hours at 37° C. To confirm the transfection of the nucleic acid into the exosomes, the transfected mixture is treated with DNase or RNase to digest unincorporated nucleic acid, and subjected to a conventional gel electrophoresis assay to determine the presence of undigested nucleic acid in the sample.

### Example 23

### Encapsulation by Electroporation

As an example, electroporation is carried out using a voltage in the range of 150 mV to 250 mV, particularly 200 mV. In another embodiment, electroporation is carried out with capacitance between 25 uT and 250 uT, particularly between 25 uT and 125 uT.

### Example 24

### Investigating Exosome's Skin Permeability

About 40 ul of the exosomes are applied per square centimeter on the surface of donated human skin. The skin is allowed to dry prior to be embedded for cryosection. The slices are prepared by covering the skin samples with a thin layer of Tissue-Tek (Sakura), and immersed for 60 seconds into 2-methylbutane cooled by liquid nitrogen. The samples are then stored at −80° C. The samples are cut perpendicular to the surface into 30 um slices using a Crypotome (Thermo Scientific), and transferred to a glass pre-treated with poly-L-Lysine. Raster image correlation spectroscopy (RICS) is used to detect the fluorescent signal of the sliced samples. The microscopy is set up as follows: a Ti:Sa laser is used for the excitation wavelength of 838 nm. The fluorescent signals is collected using bandpass filters ET Bandpass 572/35 nm and BrighLine HC 676/29 nm. The light is divided by 620-DCXXR beam splitter. The detector is Hamamatsu H7422P-40 photomultiplier tube. About 50-100 images are combined for RICS measurements. In addition, stimulated emission depletion (STED) microscopy is used to visualize the layers of skin, such as stratum corneum, in the order of 100 nm. To visualize the skin layers, an atto-488-DPPE dye is used with the excitation wavelength of 488 nm and the depletion wavelength of 592 nm.

### Example 25

### Methods of Differentiating Bone Marrow MSCs to Chondrocytes

Bone marrow mesenchymal stem cells are isolated by their ability to adhere to tissue culture plate. Bone marrow cell suspension is seeded on the culture flasks. Of the heterogeneous mixture of bone marrow cells, erythrocytes and leukocytes do not attach and thus, they can be removed during medium change. Bone marrow mesenchymal cells can be separated from bone marrow aspirate by Percoll-gradient centrifugation in accordance with methods known in the art. Human bone marrow is surgically removed from a donor. The aspirate is mixed with a complete medium in a 50-ml tube. The sample is centrifuged at 500×g for 5 min. After the sample has been centrifuged, remove the fat layer and aspirate the supernatant. Resuspend the pellet in the complete medium. To fractionate the nucleated cells including the mesenchymal stem cells, load up to 2.0×10⁸ resuspended cells on top of a preformed Percoll gradient (density 1.03-1.12 g/ml). Centrifuge at 400×g for 15 min. Pipette off the top layer or layers of cells (about 10-14 ml) and transfer them to another sterile 50-ml centrifuge tube. Add serum-containing medium to the tube for a final volume of 50 ml and centrifuge at 500×g for 5 min. Discard the supernatant, and resuspend the pellet in 10 ml of complete medium. Mesenchymal stem cells are then cultured at 37° C. in a humidified atmosphere of 95% air and 5% CO₂. Continue the culture for about 4 to 6 days until fibroblast-like cells forming loose colonies of spindle-shaped cells are visible. Once the colonies grow in size and numbers, which takes about 7 to 10 days, the cells are detached from the culture flasks and replated at 3.5-4.0×10³ cells/cm². The cells are grown to confluency, harvested, and replated at a density of 1.25×10⁶ cells/ml in chondrogenic differentiation medium. The medium comprises Dulbecco's Modified Eagle's Medium, High (4.5 g/l) Glucose supplemented with 10% ITS+Premix Tissue Culture Supplement (Becton Dickinson), 10⁻⁷ M dexamethasone (Sigma), 1 μM ascorbate-2-phosphate (Wako), 1% sodium pyruvate (Invitrogen), and 10 ng/ml transforming growth factor-beta 1 (TGF-β1). TGF-β1 is prepared by diluting a 1-μg/ml in 4 mM hydrochloric acid (HCl) and 1% bovine serum albumin. The mesenchymal stem cells are then differentiated into chondrocyte by being cultured for about 3 weeks in chondrogenic differentiation medium with daily change of medium.

### Example 26

### Method of Producing Porous Silk Fibroin

Silk polymer or silk fibroin solution is prepared as follows. B. mori cocoons are boiled for about 30 minutes in an aqueous solution of 0.02 M Na₂CO₃. The cocoons are rinsed with water, to extract the sericin proteins and the extracted silk is dissolved in an aqueous salt solution. The extracted silk is dissolved in about 9-12 M LiBr solution. The salt is consequently removed by dialysis. The silk polymer solution is formed by mixing about 5% to 35% by weight of the silk fibroin with a solvent. Preferably about 17% (w/v) of silk is used. Solvent casting and particulate leaching is performed by mixing water-solution porogen, such as NaCl, with a viscous silk polymer solution. The mixture is cast into a Teflon container where the solvent is evaporated. The result is a salt/polymer composite. The composite is immersed in water to leach out the salt, resulting in a porous three-dimensional structure.

### Example 27

### Preparation of Buffy Coat

Buffy coat can be made by centrifuging the anti-coagulated blood in a narrow test tube, then carefully removing as much as possible of the plasma without disturbing the buffy coat. Buffered 2% Glutaraldehyde is then very gently layered on top and the tube left to stand in the fridge for about a couple of hours. This gives a buffy coat which is embedded in solid plasma and can be removed from the tube with the help of a thin wooden stick or similar object. The resultant disk can then be trimmed and the pieces processed to resin for use with normal tissue. Then, the slender tube is cut with a razor blade above and below the buffy coat (on a piece of Parafilm), making a short log with open ends. Then, with a paper clip or applicator stick (depending on the diameter of the tube) the packed buffy coat is pushed out. 1% molten agar is sometimes used to keep it together. The pellet is then processed.

### Example 28

### Preparation of ECM and Compositions Comprising ECM

WI-38 human lung fibroblasts (ATCC) are cultured at the cell density of 2×10⁴ cells/cm² on the tissue culture dish (100 mm diameter) for 7 days in the DMEM supplemented with 10% FBS and 1% P/S. Once confluent, the layer is decellularized by washing the dish twice with PBS, then incubating briefly in a detergent solution containing 0.15% Triton X-100 and 10 mM NH₄OH at 37° C., and then treating with 50 U/mL DNase I and 50 μg/mL RNase A for 1 hour. After the decellularization process, ECMs are collected into centrifuge. To conjugate heparin, the collected ECM is washed with PBS and saturated with 0.05 M 2-(Nmorpholino) ethanesulfonic acid hydrate (MES) buffer (pH 5.5). 0.25% (w/v) heparin working solution is prepared by adding heparin sodium to a freshly prepared solution of 0.05 M N-(3-dimethylaminopropyl)-N′-ethylcarbodiimide hydrochloride) (EDC) and 0.06 M N-hydroxysuccinimide (NHS) in MES solution; the EDC/NHS/MES solution is vigorously mixed and left for 10 min before the use. 2 mL of heparin working solution is added to the collected ECM in a 6-well plate. They are incubated at room temperature overnight on a plate rotator. To add differentiated chondrocytes, engineered exosomes, and collagen to the heparin-conjugated ECM, differentiated cells are prepared as single cell suspension. The single cell suspension and the exosomes are mixed with the heparin-conjugated ECM in an ice-bath. Collagen (type I collagen, 3 mg/mL) is added dropwise to the mixture. Optionally, the mixture is gelatinated by incubating at 37° C. in humidified atmosphere with 5% CO₂ for 30 min. The differentiated cells are grown in a medium comprising DMEM supplemented with 2% FBS, 1% P/S, 100 nM dexamethasone, 100 μg/mL proline, 100 μg/mL pyruvate, 1% ITS+premix (6.25 μg/mL insulin, 6.25 μg/mL transferrin, 6.25 μg/mL selenium, 5.33 μg/mL linoleic acid, and 1.25 μg/mL bovine serum albumin), 50 μg/mL ascorbate-2-phosphate, and 10 ng/mL TGF-β1.

The invention claimed is:

1. A composition comprising:
1) differentiated human cells;
2) adhesive scaffold material; and
3) engineered exosomes carrying cargo molecules.

2. The composition of claim 1, wherein said cargo molecules are Col17A1 activator, apocynin, Y-27632, Col17A1, TGF-β1, TGF-β2, TGF-β3, BMP-2, BMP-3, BMP-4, BMP-5, BMP-6, BMP-7, BMP-9 or a combination thereof.

3. The composition of claim 1, wherein about 50 percent of said cargo molecules are encapsulated within said exosomes, and about 50 percent of the rest of said cargo molecules are not bound to, or encapsulated within said exosomes.

4. The composition of claim 1, wherein substantially all of said cargo molecules are encapsulated within said exosomes.

5. The composition of claim 1, wherein said exosomes are isolated from keratinocytes, thymic epithelial cells, induced pluripotent stem cell, or mesenchymal stem cells.

6. The composition of claim 1, wherein said exosomes are derived from 1OT1/2, BALB/3T3, L-M, NB4, 1A3, NIE-1 15, NG108-15, NIH3T3, NCTC, Neuro-2A, PC12, GH1, GH3, C6, L2, CHO, OHO, 6E6, PK15, LLC-PK1, ST, ESK-4, CPAE, BT, FB2, SBAC, NBL-6, COS-1, COS-7, or VV-1, SH-SY5Y, SK-N-DZ, SK-N-F1, SK-N-SH, BE(2)-C, HeLa, HEK 293, MCF-7, HepG2, HL-60, IMR-32, SW-13, OHP3, or CHPS.

7. The composition of claim 1, wherein said exosomes encapsulate said cargo by passive diffusion, sonication, saponication, heating, emulsification, freezing and thawing, or use of solvents.

8. The composition of claim 1, wherein said exosomes are isolated from cells genetically modified to express said cargo molecules.

9. The composition of claim 1, wherein said exosomes are isolated from exosome secreting cells by ultracentrifugation, ultrafiltration, size-exclusion chromatography, immunoisolations, precipitation, or microfluidics-based isolation.

10. The composition of claim 1, wherein said exosomes have Synapsin I.

11. The composition of claim 1, wherein said cargo molecules fills up about 30% of said exosomes' lumen.

12. The composition of claim 1, wherein said differentiated cells are derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, or precursor cells.

13. The composition of claim 1, wherein the amount of said differentiated cells are 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

14. The composition of claim 1, wherein said adhesive scaffold material is porous silk fibroin wherein said scaffold has pores of 50 to 1,000 microns in diameter, a density of 20-200 m/ml, a porosity from 50-99.5%, and a compressive modulus of at least 100 kPa.

15. The composition of claim 1, wherein said adhesive scaffold material is cell-based extracellular matrix or buffy coat.

16. The composition of claim 1, wherein said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

17. The composition of claim 1, wherein said composition further comprise a buffered solution selected from the group consisting of phosphate-buffered saline, Hanks balance salt solution, or saline.

18. The composition of claim 1, wherein said cargo molecules are released in biphasic manner in which about 50 percent of the cargo molecules are released during the first phase and the other 50 percent of the cargo molecules are released during the second phase.

19. The composition of claim 1, wherein the amount of said exosomes in a package is about 4 mg.

20. A composition comprising 1×10⁶ human chondrocytes; exosomes carrying TGF-β1, and an adhesive scaffold material.

21. A composition of claim 20, wherein said composition further comprising TGF-β1.

22. A composition of claim 20, wherein said scaffold material is buffy coat.

23. A composition comprising 1×10⁶ human fibroblasts; exosomes carrying apocynin, and an adhesive scaffold material.

24. A composition of claim 23, wherein said scaffold material is collagen matrix.

25. A method comprising:
1) Differentiating mammalian cells in vitro;
2) Culturing exosome secreting cells;
3) Modifying said exosome-producing cells or exosomes secreted therefrom to carry cargo molecules in said exosome;
4) Isolating said exosomes; and
5). Formulating an injectable composition comprising said differentiated cells, said exosomes, and adhesive scaffold material.

26. The method of claim 25, wherein said cargo molecules are Col17A1 activator, apocynin, Y-27632, Col17A1, TGF-β1, TGF-β2, TGF-β3, BMP-2, BMP-3, BMP-4, BMP-5, BMP-6, BMP-7, BMP-9, or a combination thereof.

27. The method of claim 25, wherein said injectable composition has about 50 percent of said cargo molecules encapsulated within said exosomes, and about 50 percent of the rest of said cargo molecules not bound to, or encapsulated within said exosomes.

28. The method of claim 25, wherein substantially all of said cargo molecules are encapsulated within said exosomes.

29. The method of claim 25, wherein said mammalian cells are keratinocytes, thymic epithelial cells, induced pluripotent stem cell, or mesenchymal stem cells.

30. The method of claim 25, wherein said modifying is passive diffusion, sonication, saponication, heating, emulsification, freezing and thawing, or use of solvents.

31. The method of claim 25, wherein said modifying is genetically modifying said exosome secreting cells to express said cargo molecules.

32. The method of claim 25, wherein said isolating is ultracentrifugation, ultrafiltration, size-exclusion chromatography, immunoisolations, precipitation, or microfluidics-based isolation.

33. The method of claim 25, wherein said exosomes have Synapsin I.

34. The method of claim 25, wherein said cargo molecules fills up about 30% of the lumen of said exosomes.

35. The method of claim 25, wherein said differentiated cells are derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, or precursor cells.

36. The method of claim 25, wherein the amount of said differentiated cells are 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

37. The method of claim 25, wherein said adhesive scaffold material is porous silk fibroin wherein said scaffold has pores of 50 to 1,000 microns in diameter, a density of 20-200 m/ml, a porosity from 50-99.5%, and a compressive modulus of at least 100 kPa.

38. The method of claim 25, wherein said adhesive scaffold material is cell-based extracellular matrix or buffy coat.

39. The method of claim 25, wherein said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

40. The method of claim 25, wherein said injectable composition further comprise a buffered solution selected from the group consisting of phosphate-buffered saline, Hanks balance salt solution, or saline.

41. The method of claim 25, wherein said cargo molecules are released in biphasic manner in which about 50 percent of the cargo molecules are released during the first phase and the other 50 percent of the cargo molecules are released during the second phase.

42. The method of claim 25, wherein the amount of said exosomes in said injectable composition is about 4 mg.

43. A method of identifying compositions carrying therapeutic amounts of engineered exosomes, differentiated cells, and adhesive scaffold material comprising:
1) Culturing exosome-secreting cells;
2) Modifying said cells or exosomes secreted therefrom to carry cargo molecules in said exosome;
3) Isolating said exosomes;
4) Formulating an injectable composition comprising said exosomes, differentiated cells, adhesive scaffold material, and pharmaceutically acceptable excipients;
5) Administering said formulation to a human subject;
6) Examining the clinical responses of a human subject; and
7) Identifying formulations with desired therapeutic effects.

44. The method of claim 43, wherein said cargo molecules are Col17A1 activator, apocynin, Y-27632, Col17A1, TGF-β1, TGF-β2, TGF-β3, BMP-2, BMP-3, BMP-4, BMP-5, BMP-6, BMP-7, BMP-9, or a combination thereof.

45. The method of claim 43, wherein said injectable composition has about 50 percent of said cargo molecules encapsulated within said exosomes, and about 50 percent of the rest of said cargo molecules not bound to, or encapsulated within said exosomes.

46. The method of claim 43, wherein substantially all of said cargo molecules are encapsulated within said exosomes.

47. The method of claim 43, wherein said culturing is growing keratinocytes, thymic epithelial cells, induced pluripotent stem cell, or mesenchymal stem cells in vitro.

48. The method of claim 43, wherein said modifying is passive diffusion, sonication, saponication, heating, emulsification, freezing and thawing, or use of solvents.

49. The method of claim 43, wherein said modifying is genetically modifying said exosome secreting cells to express said cargo molecules.

50. The method of claim 43, wherein said isolating is ultracentrifugation, ultrafiltration, size-exclusion chromatography, immunoisolations, precipitation, or microfluidics-based isolation.

51. The method of claim 43, wherein said exosomes have Synapsin I.

52. The method of claim 25, wherein said cargo molecules fills up about 30% of the lumen of said exosomes.

53. The method of claim 43, wherein said differentiated cells are derived from smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, stem cells, progenitor cells, or precursor cells.

54. The method of claim 43, wherein the amount of said differentiated cells are 0.5×10⁵ cells, 1×10⁵ cells, 1×10⁶ cells, 1×10⁷ cells, 1×10⁸ cells, 2×10⁵ cells, 2×10⁶ cells, 2×10⁷ cells, 2×10⁸ cells, 3×10⁵ cells, 3×10⁶ cells, 3×10⁷ cells, 3×10⁸ cells, 5×10⁵ cells, 5×10⁶ cells, 5×10⁷ cells, 5×10⁸ cells, 7×10⁵ cells, 7×10⁶ cells, 7×10⁷ cells, 7×10⁸ cells, 8.5×10⁵ cells, 8.5×10⁶ cells, 8.5×10⁷ cells, or 8.5×10⁸ cells.

55. The method of claim 43, wherein said adhesive scaffold material is porous silk fibroin wherein said scaffold has pores of 50 to 1,000 microns in diameter, a density of 20-200 m/ml, a porosity from 50-99.5%, and a compressive modulus of at least 100 kPa.

56. The method of claim 43, wherein said adhesive scaffold material is cell-based extracellular matrix or buffy coat.

57. The method of claim 43, wherein said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

58. The method of claim 43, wherein said injectable composition further comprise a buffered solution selected from the group consisting of phosphate-buffered saline, Hanks balance salt solution, or saline.

59. The method of claim 43, wherein said cargo molecules are released in biphasic manner in which about 50 percent of the cargo molecules are released during the first phase and the other 50 percent of the cargo molecules are released during the second phase.

60. The method of claim 43, wherein the amount of said exosomes in said injectable composition is about 4 mg.

61. The method of claim 43, wherein said clinical response is measuring VAS, KOOS, or IKDC scoring.

62. The method of claim 43, wherein said desired effect is a change in clinical response recorded by at least one unit of measure employed in a scoring method.
